(19) United States
(12) Patent Application Publication
(10) Pub. No.: US 2022/0371986 A1
(43) Pub. Date: Nov. 24, 2022

(54) METHOD TO GENERATE BIOCHEMICALLY REACTIVE AMINO ACIDS

(71) Applicant: The Regents of the University of California, Oakland, CA (US)

(72) Inventor: Lei Wang, San Francisco, CA (US)

(21) Appl. No.: 17/599,907

(22) PCT Filed: Apr. 3, 2020
(86) PCT No.: PCT/US2020/026704

§ 371 Date: Sep. 29, 2021

Related U.S. Application Data
(60) Provisional application No. 62/829,300, filed on Apr. 4, 2019.

(51) Int. Cl. C07C 227/16; C07K 2/00; C12P 21/02; C12N 1/20; C12N 15/70; C12N 9/00

(52) U.S. Cl. CPC C07C 227/16; C07K 2/00; C12P 21/02; C12N 1/20; C12N 15/70; C12N 9/93; C12Y 601/01026; C12N 2800/101

(57) ABSTRACT

Provided herein are, inter alia, methods of forming chemically reactive amino acids and methods of using same.

### CROSS-REFERENCE TO RELATED APPLICATIONS

This application claims priority to U.S. Application No. 62/829,300 filed Apr. 4, 2019, the disclosure of which is incorporated by reference herein in its entirety.

### STATEMENT AS TO RIGHTS TO INVENTIONS MADE UNDER FEDERALLY SPONSORED RESEARCH AND DEVELOPMENT

This invention was made with government support under grant no. R01 GM118384 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-639001WO_SEQUENCE_LISTING_ST25, created on Apr. 1, 2020, 15,663 bytes, machine format IBM-PC, MS-Windows operating system, is hereby incorporated by reference.

### BACKGROUND

Expansion of the genetic code with unnatural amino acids (Uaas) has significantly increased the chemical space available to proteins for exploitation. However, due to the inherent limitation of translational machinery and the required compatibility with biological settings, function groups introduced via Uaas to date are restricted to chemically inert, bioorthogonal, or latent bioreactive groups. Through engineering orthogonal components for protein translation, unnatural amino acids (Uaas) have been genetically encoded in various cells and model organisms. See Wang et al, Science, 292(5516):498-500 (2001); Wang et al, Angew. Chem. Int. Ed. Engl., 44(1):34-66 (2005); Liu et al, Annu. Rev. Biochem., 79(1):413-444 (201); Wang, Acc. Chem. Res., 50(11):2767-2775 (2017); Chen, et al, Cell Res., 27(2):294-297 (2017). To be compatible with biological settings, side chains of these encoded Uaas are mainly chemically inert or bioorthogonal. See Wang, Angew. Chem. Int. Ed. Engl. 2005, 44 (1), 34-66; Liu et al, Annu. Rev. Biochem. 2010, 79 (1), 413-444; Wang et al, Annu. Rev. Biophys. Biomol. Struct. 2006, 35 (1), 225-249. A recent breakthrough is the encoding of latent bioreactive Uaas, which are unreactive inside cells but once incorporated into proteins able to form covalent bonds with natural amino acid residues in proximity. See Xiang et al, Nat. Methods 2013, 10 (9), 885-888; Xiang et al, Angew. Chem. Int. Ed. Engl. 2014, 53, 2190-2193; Furman et al, J. Am. Chem. Soc. 2014, 136 (23), 8411-8417; Wang, N. Biotechnol., 2017, 38 (Pt A), 16-25. Nonetheless, it remains infeasible to selectively introduce chemically reactive Uaas into proteins in live cells, because the chemical reactivity of the Uaa may interfere with protein translation and other biological processes. Nature, on the other hand, has installed the reactive dehydroalanine (Dha) and dehydrobutyrine (Dhb) into proteins through enzymatic posttranslational modifications, which are used to create unique intra-protein bridges in lantipeptides and thiopeptides possessing antimicrobial and antitumor activities. See Li et al, Science 2007, 315 (5814), 1000-1003; Repka et al, Chem. Rev. 2017, 117 (8), 5457-5520. Through chemical conversion Dha and Dhb can also be generated in vitro, and the unique structure and reactivity of a,b-unsaturated carbonyl moiety in Dha have been harnessed for chemical mutagenesis and chemical installation of a broad range of posttranslational modifications, providing an invaluable route for studying proteins. See Seebeck et al, J. Am. Chem. Soc. 2006, 128 (22), 7150-7151; Wang et al, Angew. Chem. Int. Ed. 2007, 46 (36), 6849-6851; Guo et al, Angew. Chem. Int. Ed. Engl. 2008, 47 (34), 6399-6401; Wang et al, Biochemistry 2012, 51 (26), 5232-5234; Wright et al, Science 2016, 354 (6312), aag1465-aag1465; Yang et al, Science 2016, 354 (6312), 623-626; Freedy et al, J. Am. Chem. Soc. 2017, 139 (50), 18365-18375; Dadova et al, Curr. Opin. Chem. Biol. 2018, 46, 71-81; de Bruijn et al, Chemistry 2018, 24 (48), 12728-12733. However, due to the cellular incompatibility of reagents and conditions used for chemical conversion, methods reported to date cannot generate Dha or Dhb in vivo.

Provided herein are, inter alia, solutions to these and other problems and needs in the art.

### SUMMARY

The disclosure provides methods of converting an amino acid to a chemically reactive amino acid by contacting an FSY protein with the amino acid; thereby converting the amino acid to a chemically reactive amino acid. In aspects, the methods comprise converting serine to dehydroalanine. In aspects, the methods comprise converting threonine to dehydrobutyrine. In aspects, the methods further comprise glycosylating the chemically reactive amino acid. In aspects, the reaction occurs within a cell.

The disclosure provides methods of converting an amino acid to a chemically reactive amino acid by the steps of: (i) contacting a protein, a pyrrolysyl-tRNA synthetase, a tRNA^{Pyl}, and a fluorosulfate-L-tyrosine, thereby forming the FSY protein; and (ii) contacting the FSY protein with the amino acid; thereby converting the amino acid to a chemically reactive amino acid. In aspects, the methods comprise converting serine to dehydroalanine. In aspects, the methods comprise converting threonine to dehydrobutyrine. In aspects, the methods further comprise glycosylating the chemically reactive amino acid. In aspects, the reaction occurs within a cell.

The disclosure provides proteins comprising: (i) fluorosulfate-L-tyrosine, and (ii) serine, threonine, or a combination thereof proximal to the fluorosulfate-L-tyrosine. In aspects, the proteins comprise: (i) fluorosulfate-L-tyrosine, and (ii) dehydroalanine, dehydrobutyrine, or a combination thereof proximal to the fluorosulfate-L-tyrosine. In aspects, the proteins comprise: (i) tyrosine, and (ii) dehydroalanine, dehydrobutyrine, or a combination thereof proximal to the tyrosine.

The disclosure provides protein complexes comprising: (i) a first protein comprising fluorosulfate-L-tyrosine, and (ii) a second protein comprising serine, threonine, or a combination thereof; wherein the fluorosulfate-L-tyrosine in the first protein is proximal to the serine, threonine, or the combination thereof in the second protein. In aspects, the protein complex comprises: (i) a first protein comprising fluorosulfate-L-tyrosine, and (ii) a second protein comprising dehydroalanine, dehydrobutyrine, or a combination thereof; wherein the fluorosulfate-L-tyrosine in the first protein is proximal to the dehydroalanine, dehydrobutyrine, or the combination thereof in the second protein. In aspects, the protein complex comprises: (i) a first protein comprising tyrosine, and (ii) a second protein comprising dehydroalanine, dehydrobutyrine, or a combination thereof; wherein the tyrosine in the first protein is proximal to the dehydroalanine, dehydrobutyrine, or the combination thereof in the second protein.

These and other embodiments and aspects of the disclosure are provided in more detail herein.

### BRIEF DESCRIPTION OF THE DRAWINGS

FIG. 1 is a diagram showing that GECCO site-selectively introduced chemically reactive amino acids into proteins in vivo. The latent bioreactive Uaa FSY reacts with a nearby Ser or Thr via proximity-enabled reactivity, selectively converting the latter into Dha or Dhb.

FIGS. 2A-2J show the generation of Dha and Dhb on proteins via intermolecular GECCO in E. coli. FIG. 2A: Structure of Afb-Z complex (PDB: 1LP1) showing two proximal sites for placing FSY and the target Ser. FIGS. 2B-2C: Tandem mass spectra identifying Ser and Dha at site 7 of the Afb protein. FIGS. 2D-2E: Tandem mass spectra identifying FSY and Tyr at site 24 of the Z protein. FIG. 2F: Structure of Afb-Z complex (PDB: 1LP1) showing two proximal sites for placing FSY and the target Thr. FIGS. 2G-2H: Tandem mass spectra identifying Thr and Dhb at site 7 of the Afb protein. FIGS. 2I-2J: Tandem mass spectra identifying FSY and Tyr at site 24 of the Z protein.

FIGS. 3A-3C show the generation of Dha on sfGFP via intramolecular GECCO in E. coli. FIG. 3A: Crystal structure of sfGFP (PDB: 2B3P) showing site Tyr182 for FSY incorporation to target Ser introduced at site Glu184 on the β-strand. FIGS. 3B-3C: Tandem MS spectra of sfGFP (182FSY/184Ser) expressed in E. coli identifying 182FSY/184Ser (FIG. 3B) and 182Tyr/184Dha (FIG. 3C).

FIGS. 4A-4F show the generation of Dha on Afb via intramolecular GECCO in E. coli. FIGS. 4A-4B: Tandem mass spectra identifying Dha at Ser-1 (FIG. 4A) and Ser10 (FIG. 4B) of the Afb protein. FIGS. 4C-4D: Histogram of C_{β}-C_{β} distances of Ser-1 and Asp37 (FIG. 4C) and of Ser10 and Asp37 (FIG. 4D) in 4,525 low energy models of ab initio folded Afb. FIGS. 4E-4F: Representative models from ab initio folding of Afb showing Asp37 close to Ser-1 (FIG. 4E) and close to Ser10 (FIG. 4F). The left-handed (gold) structure in (FIG. 4E) is the aligned Afb backbone of 1LP1.

FIGS. 5A-5C show labeling Dha-containing sfGFP with 1-thiol-GlcNAc. FIG. 5A: Scheme showing the structure of 1-thiol-GlcNAc and its reaction with Dha. Western blot (FIG. 5B) and tandem MS analysis (FIG. 5C) of the reaction product confirmed successful labeling of Dha with GlcNAc.

FIG. 6 is a diagram showing that GECCO site-selectively introduced chemically reactive amino acids into proteins in vivo. The latent bioreactive Uaa FSY reacts with a nearby Ser or Thr via proximity-enabled reactivity, selectively converting the latter into Dha or Dhb. Dha is labeled with a thiol-derivatized saccharide to produce a glycoprotein mimetics.

### DETAILED DESCRIPTION

Expansion of the genetic code with unnatural amino acids (Uaas) has significantly increased the chemical space available to proteins for exploitation. Due to the inherent limitation of translational machinery and the required compatibility with biological settings, function groups introduced via Uaas to date are restricted to chemically inert, bioorthogonal, or latent bioreactive groups. To break this barrier, this disclosure provides a new strategy enabling the specific incorporation of biochemically reactive amino acids into proteins. A latent bioreactive amino acid is genetically encoded at a position proximal to the target natural amino acid; they react via proximity-enabled reactivity, selectively converting the latter into a reactive residue in situ. Using this Genetically Encoded Chemical COnversion (GECCO) strategy and harnessing the sulfur-fluoride exchange (SuFEx) reaction between fluorosulfate-L-tyrosine and serine or threonine, the reactive dehydroalanine and dehydrobutyrine are site-specifically generated into proteins. GECCO works both inter- and intramolecularly, and is compatible with various proteins. The resultant dehydroalanine-containing protein was further labeled with thiol-saccharide to generate glycoprotein mimetics. GECCO represents a new solution for selectively introducing biochemically reactive amino acids into proteins and is expected to open new avenues for exploiting chemistry in live systems for biological research and engineering.

The inventors recently developed an orthogonal tRNAPyl/FSYRS pair that genetically incorporates the unnatural amino acid FSY in response to the amber stop codon UAG into proteins in E. coli and mammalian cells. See Wang et al, J. Am. Chem. Soc., 140:4995-4999 (2018). The incorporated FSY was found to react with Lys, His, and Tyr in proximity through sulfur-fluoride exchange (SuFEx) reaction, forming covalent protein crosslinks in vivo. However, no crosslinking was detected between FSY and serine or threonine on SDS-PAGE. In contrast, arylfluorosulfate installed on chemical probes was able to react with Lys, Tyr, and Ser within a positively charged binding pocket of the specifically bound protein, and the resultant arylfluorosulfate-Ser adduct was found to partially hydrolyze to Dha, but what occurred to the arylfluorosulfate warhead remains uncharacterized. See Chen et al, J. Am. Chem. Soc., 138(23):7353-7364 (2016); Mortenson et al, J. Am. Chem. Soc., 140(1):200-210 (2018); Fadeyi et al, ACS Chem. Biol., 12(8):2015-2020 (2017). Prompted by these findings, the inventors investigated whether proximal FSY/serine and proximal FSY/threonine incorporated into proteins (instead of on small molecules) would react, whether a positively charged microenvironment was necessary, and what the products would be. The inventors thus incorporated FSY/serine and FSY/threonine in different protein contexts without positively charged residues nearby, and characterized their identity using high resolution tandem MS. The results are described herein.

### Definitions

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), small interfering RNA (siRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In aspects, the amino acid side chain may be a non-natural amino acid side chain. In aspects, the amino acid side chain is H,

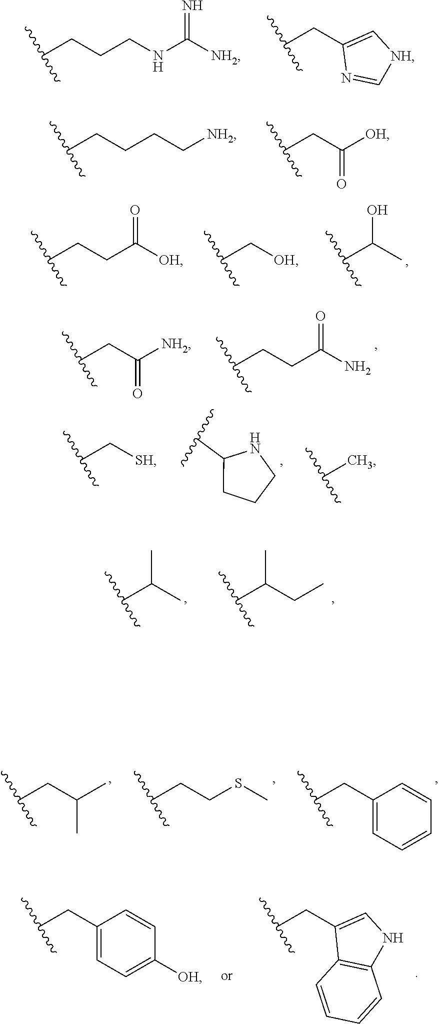

The term “non-natural amino acid side chain” or “unnatural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]hept-5-ene-2-carboxylic acid hydrochloride, cis-2-aminocycloheptane-carboxylic acid hydrochloride, cis-6-amino-3-cyclohexene-1-carboxylic acid hydrochloride, cis-2-amino-2-methylcyclohexanecarboxylic acid hydrochloride, cis-2-amino-2-methylcyclopentane-carboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine.

In embodiments, the unnatural amino acid is fluorosulfate-L-tyrosine (FSY) having the following Formula (TV

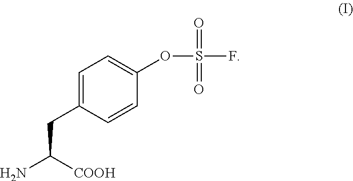 [SMILES: N[C@@H](Cc1ccc(OS(=O)(=O)F)cc1)C(=O)O]

In embodiments, the unnatural amino acid side chain is a moiety of Formula (II):

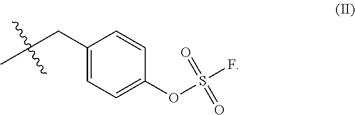 [SMILES: *Cc1ccc(OS(=O)(=O)F)cc1]

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M). (see, e.g., Creighton, Proteins (1984)).

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to Ala302 of the PylRS protein when the selected residue occupies the same essential spatial or other structural relationship as Ala302 in the PylRS protein. In embodiments, where a selected protein is aligned for maximum homology with the PylRS protein, the position in the aligned selected protein aligning with Ala302 is said to correspond to Ala302. Instead of a primary sequence alignment, a three dimensional structural alignment can also be used, e.g., where the structure of the selected protein is aligned for maximum correspondence with the PylRS protein and the overall structures compared. In this case, an amino acid that occupies the same essential position as Ala302 in the structural model is said to correspond to the Ala302 residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, or at least 65%, 70%, 75%, 80%, 85%, 90%, 91%, 92%, 93%, 94%, 95%, 96%, 97%, 98%, 99% identity over a specified region, when compared and aligned for maximum correspondence over a comparison window or designated region) as measured using a BLAST or BLAST 2.0 sequence comparison algorithms with default parameters described below, or by manual alignment and visual inspection (e.g., NCBI web site ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “biomolecule” as used herein refers to large macromolecules such as, for example, proteins, carbohydrates, lipids, and nucleic acids, as well as small molecules such as, for example, primary and secondary metabolites. In aspects, the term biomolecule refers to a protein. In aspects, the term biomolecule refers to a nucleic acid or a carbohydrate.

The term “biomolecule moiety” as used herein refers to biomolecules, including large macromolecules such as, for example, proteins, carbohydrates, lipids, and nucleic acids, as well as small molecules such as, for example, primary and secondary metabolites. Thus, in embodiments, the biomolecule moiety is a peptidyl moiety, a carbohydrate moiety, a lipid moiety or a nucleic acid moiety. Biomolecule moieties may form part of a molecule (e.g., biomolecule). For example, biomolecule moieties may form part of a biomolecule conjugate, where the biomolecule conjugate includes two or more biomolecule moieties. In embodiments, the biomolecule conjugate includes two or more biomolecule moieties conjugated via a bioconjugate linker.

The term “peptidyl moiety” as used herein refers to a protein, protein fragment, or peptide. The peptidyl moiety may also be substituted with additional chemical moieties.

The term “carbohydrate moiety” as used herein refers to carbohydrates, for example, polyhydroxy aldehydes, ketones, alcohols, acids, their simple derivatives and their polymers having linkages of the acetal type. The carbohydrate moiety may also be substituted with additional chemical moieties.

The term “nucleic acid moiety” as used herein refers to nucleic acids, for example, DNA, and RNA. The nucleic acid moiety may also be substituted with additional chemical moieties.

The term “pyrrolysyl-tRNA synthetase” refers to an enzyme (including homologs, isoforms, and functional fragments thereof) with pyrrolysyl-tRNA synthetase activity. Pyrrolysyl-tRNA synthetase is an aminoacyl-tRNA synthetase that catalyzes the reaction necessary to attach α-amino acid pyrrolysine to the cognate tRNA (tRNA^{pyl}), thereby allowing incorporation of pyrrolysine during proteinogenesis at amber stop codons (i.e., UAG). The term includes any recombinant or naturally-occurring form of pyrrolysyl-tRNA synthetase or variants, homologs, or isoforms thereof that maintain pyrrolysyl-tRNA synthetase activity (e.g. within at least 30%, 40%, 50%, 60%, 70%, 80%, 90%, 95%, or 100% activity compared to wild-type pyrrolysyl-tRNA synthetase). In aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring pyrrolysyl-tRNA synthetase. In aspects, the pyrrolysyl-tRNA synthetase includes the sequence set forth by SEQ ID NO:3. In aspects, the pyrrolysyl-tRNA synthetase is the sequence set forth by SEQ ID NO:3. In aspects, the pyrrolysyl-tRNA synthetase is a mutant pyrrolysyl-tRNA synthetase. In aspects, the mutant pyrrolysyl-tRNA synthetase includes the sequence set forth by SEQ ID NO:1. In aspects, the mutant pyrrolysyl-tRNA synthetase is the sequence set forth by SEQ ID NO:1. In aspects, the mutant pyrrolysyl-tRNA synthetase is encoded by the sequence set forth by SEQ ID NO:2. In aspects, the mutant pyrrolysyl-tRNA synthetase catalyzes the attachment of fluorosulfate-L-tyrosine (FSY) to a tRNA^{pyl}.

The terms “tRNA^{Pyl}” and “rTNA^{Pyl}_{CUA}” and “tRNA^{Pyl}_{CUA}” (i.e., tRNA(superscript Pyl)(subscript CUA)) are used interchangeably and all refer to a single-stranded RNA molecule containing about 70 to 90 nucleotides which fold via intrastrand base pairing to form a characteristic cloverleaf structure that carries a specific amino acid (e.g., pyrrolysine, FSY) and matches it to its corresponding codon (i.e., a complementary to the anticodon of the tRNA) on an mRNA during protein synthesis. In tRNA^{Py}, the anticodon is CUA. Anticodon CUA is complementary to amber stop codon UAG. The abbreviation “Pyl” of tRNA^{Py} stands for pyrrolysine and the “CUA” of tRNA^{Py} refers to its anticodon CUA. In embodiments, tRNA^{Py} is attached to FSY.

The term “substrate-binding site” as used herein refers to residues located in the enzyme active site that form temporary bonds or interactions with the substrate. In aspects, the substrate-binding site of pyrrolysyl-tRNA synthetase refers to residues located in the active site of pyrrolysyl-tRNA synthetase that form temporary bonds or interactions with the amino acid substrate. In aspects, the substrate-binding site of pyrrolysyl-tRNA synthetase includes one or more of the following residues: alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO: 3.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. The terms “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions. Some viral vectors are capable of targeting a particular cells type either specifically or non-specifically. Exemplary vectors that can be used include, but are not limited to, pEvol vector, pMP vector, pET vector, pTak vector, pBad vector.

The term “complex” refers to a composition that includes two or more components, where the components bind together to make a functional unit. In aspects, a complex described herein include a mutant pyrrolysyl-tRNA synthetase described herein and an amino acid substrate (e.g., FSY). In aspects, a complex described herein includes a mutant pyrrolysyl-tRNA synthetase described herein and a tRNA (e.g., tRNA^{Py}). In aspects, a complex described herein includes a mutant pyrrolysyl-tRNA synthetase described herein, an amino acid substrate (e.g., FSY) and a tRNA (e.g., tRNA^{Py}). In aspects, a complex described herein includes at least two components selected from the group consisting of a mutant pyrrolysyl-tRNA synthetase described herein, an amino acid substrate (e.g., FSY), a polypeptide containing FSY, and a tRNA (e.g., tRNA^{Py}).

The term “protein complex” refers to a composition that includes two or more proteins, where the proteins are proximal to each other but not bound together; the proteins are covalently bound together; or the proteins are ionically bound together. In aspects, the proteins are proximal to each other but not bound together. In aspects, the proteins are covalently bonded together. In aspects, proteins are ionically bonded together. In aspects, the proteins are covalently and ionically bonded together. In aspects, a first protein in the protein complex comprises fluorosulfate-L-tyrosine, and a second protein in the protein complex comprises serine, threonine, or a combination thereof. In aspects, the fluorosulfate-L-tyrosine in the first protein is proximal to the serine and/or threonine in the second protein. In aspects “proximal” means that the FSY in the first protein and the serine and/or threonine in the second protein are close enough to each other for a chemical reaction to occur between the FSY protein and the serine and/or threonine. In aspects, the chemical reaction is a SuFEx reaction. In aspects, the FSY in the first protein converts the serine in the second protein to dehydroalanine. In aspects, the FSY in the first protein converts the threonine in the second protein to dehydrobutyrine. In aspects, the FSY converts to tyrosine after the chemical reaction converting the serine and/or threonine to dehydroalanine and/or dehydrobutyrine, respectively.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In aspects, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Contacting” is used in accordance with its plain ordinary meaning and refers to the process of allowing at least two distinct species (e.g. chemical compounds including amino acids, proteins, peptides, biomolecules, or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be biomolecule moieties as described herein. In some embodiments, contacting includes allowing two proteins as described herein to interact.

The symbol “ [symbol] ” or “-” denotes the point of attachment of a chemical moiety to the remainder of a molecule or chemical formula.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The terms “fluorosulfate-L-tyrosine” and “FSY” refer to the unnatural amino acid having the structure of Formula (I):

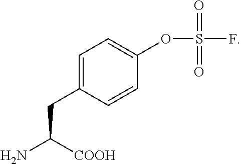 [SMILES: N[C@@H](Cc1ccc(OS(=O)(=O)F)cc1)C(=O)O]

FSY comprises the amino acid side chain of Formula (II):

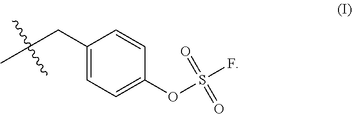 [SMILES: *Cc1ccc(OS(=O)(=O)F)cc1]

The term “FSY biomolecule” refers to a biomolecule comprising the FSY unnatural amino acid and/or the amino acid side chain thereof.

The term “FSY protein” refers to a protein comprising the FSY unnatural amino acid and/or the amino acid side chain thereof.

The term “dehydroalanine” or “Dha” refers to the chemically reactive amino acid residue having the structure of Formula (III):

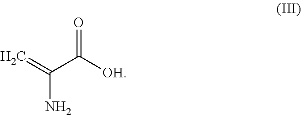

Dehydroalanine can be formed from serine by a click chemistry reaction (e.g., SuFEx).

The term “dehydrobutyrine” or “Dhb” refers to the chemically reactive amino acid residue having the structure of Formula (IV):

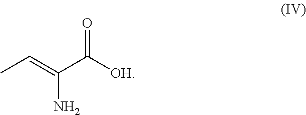

Dehydrobutyrine can be formed from threonine by a click chemistry reaction (e.g., SuFEx).

The term “sulfur-fluoride exchange reaction” or “SuFEx” refers to a type of click chemistry as described in detail by, e.g., Dong et al, Angewandte Chemie, 53(36):9340-9448 (2014); and Wang et al, J. Am. Chem. Soc., 140(15):4995-4999 (2018). The term “proximally-enabled” SuFEx refers to the sulfur-fluoride exchange reaction occurring when the reactive species are proximal to each other, i.e., spatially close enough for the SuFEx reaction to occur. The proximity may occur within a single biomolecule (e.g., protein) or between two different biomolecules (e.g., proteins). The skilled artisan could readily determine whether the reactive species are sufficiently proximal for the reaction to occur, e.g., sulfur-fluoride exchange reaction between FSY and serine and/or threonine to form the chemically reactive species of dehydroalanine and/or dehydrobutyrine, respectively.

In embodiments, the term “proximal” means that two compounds (e.g., biomolecules, proteins, peptides, amino acids) are within 1 to 20 amino acids of each other. In aspects “proximal” means that two compounds (e.g., biomolecules, proteins, peptides, amino acids) are within 1 to 15 amino acids of each other. In aspects “proximal” means that two compounds (e.g., biomolecules, proteins, peptides, amino acids) are within 1 to 10 amino acids of each other. In aspects “proximal” means that two compounds (e.g., biomolecules, proteins, peptides, amino acids) are within 1 to 9 amino acids of each other. In aspects “proximal” means that two compounds (e.g., biomolecules, proteins, peptides, amino acids) are within 1 to 8 amino acids of each other. In aspects “proximal” means that two compounds (e.g., biomolecules, proteins, peptides, amino acids) are within 1 to 7 amino acids of each other. In aspects “proximal” means that two compounds (e.g., biomolecules, proteins, peptides, amino acids) are within 1 to 6 amino acids of each other. In aspects “proximal” means that two compounds (e.g., biomolecules, proteins, peptides, amino acids) are within 1 to 5 amino acids of each other. In aspects “proximal” means that two compounds (e.g., biomolecules, proteins, peptides, amino acids) are within 1 to 4 amino acids of each other. In aspects “proximal” means t that two compounds (e.g., biomolecules, proteins, peptides, amino acids) are within 1 to 3 amino acids of each other. In aspects “proximal” means that two compounds (e.g., biomolecules, proteins, peptides, amino acids) are within 1 to 2 amino acids of each other. In aspects “proximal” means that two compounds (e.g., biomolecules, proteins, peptides, amino acids) are within 2 amino acids of each other. In aspects “proximal” means that two compounds (e.g., biomolecules, proteins, peptides, amino acids) are within 1 amino acids of each other. In aspects “proximal” means that two compounds (e.g., biomolecules, proteins, peptides, amino acids) are adjacent (e.g., but not covalently bonded together). In aspects, “proximal” means up to about 25 angstroms. In aspects, “proximal” means up to about 20 angstroms. In aspects, “proximal” means up to about 15 angstroms. In aspects, “proximal” means up to about 10 angstroms. In aspects, “proximal” means up to about 5 angstroms. In aspects, “proximal” means from about 0.1 angstroms to about 25 angstroms. In aspects, “proximal” means from about 0.1 angstroms to about 20 angstroms. In aspects, “proximal” means from about 0.1 angstroms to about 15 angstroms. In aspects, “proximal” means from about 0.1 angstroms to about 12 angstroms. In aspects, “proximal” means from about 0.1 angstroms to about 10 angstroms. In aspects, “proximal” means from about 0.1 angstroms to about 8 angstroms. In aspects, “proximal” means from about 0.1 angstroms to about 6 angstroms. In aspects, “proximal” means from about 0.1 angstroms to about 5 angstroms. In aspects, “proximal” means from about 0.1 angstroms to about 4 angstroms. In aspects, “proximal” means from about 1 angstrom to about 25 angstroms. In aspects, “proximal” means from about 1 angstrom to about 20 angstroms. In aspects, “proximal” means from about 1 angstrom to about 15 angstroms. In aspects, “proximal” means from about 1 angstrom to about 12 angstroms. In aspects, “proximal” means from about 1 angstrom to about 10 angstroms. In aspects, “proximal” means from about 1 angstrom to about 8 angstroms. In aspects, “proximal” means from about 1 angstrom to about 6 angstroms. In aspects, “proximal” means from about 1 angstrom to about 5 angstroms. In aspects, “proximal” means from about 1 angstroms to about 4 angstroms.

Biomolecules

Provided herein are biomolecules formed through the interaction of latent bioreactive unnatural amino acids with naturally occurring amino acids. Fluorosulfate-L-tyrosine (FSY), a latent bioreactive unnatural amino acid, facilitates formation of chemically reactive amino acids with proximal target amino acid residues (e.g., serine, threonine) by undergoing a click chemistry reaction (e.g., sulfur-fluoride exchange reaction (SuFEx)). For example, FSY may be inserted into or replace an amino acid in a naturally occurring protein, thereby endowing the protein with the ability to form a chemically reactive amino acid with proximally positioned target amino acid residues (e.g., serine, threonine) on the protein itself or with proteins it naturally interacts with. FSY may be used to facilitate the formation of chemically reactive amino acids in proteins and within proteins in both in vitro and in vivo conditions. As such, the latent bioreactive unnatural amino acid FSY is useful for forming chemically reactive amino acid residues that can be further chemically modified, as desired.

FSY, as a latent bioreactive unnatural amino acid, has shown excellent chemical functionality (i.e., superior properties) compared to previously described bioreactive unnatural amino acids. For example, FSY is stable, nontoxic and nonreactive inside cells, yet when placed in proximity to target amino acid residues it becomes reactive under cellular conditions. FSY is able to react with serine and threonine specifically with great selectivity via proximity-enabled SuFEx reaction within and between proteins under physiological conditions. No bioreactive unnatural amino acid has been reported that is nontoxic inside cells and is able to form chemically reactive amino acid residues, while then reverting to a chemically inactive amino acid, e.g., FSY converts to tyrosine following the formation of the chemically reactive amino acid residues.

Provided herein are biomolecules comprising one or more latent bioreactive unnatural amino acids. In aspects, the biomolecule is a protein, a nucleic acid, or a carbohydrate. In aspects, the biomolecule is a protein. In aspects, the latent bioreactive unnatural amino acid is fluorosulfate-L-tyrosine (FSY) having the Formula (I):

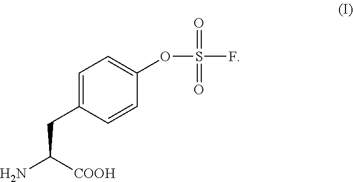 [SMILES: N[C@@H](Cc1ccc(OS(=O)(=O)F)cc1)C(=O)O]

In aspects, the biomolecule is a protein comprising the FYS unnatural amino acid (e.g., an “FSY protein”). In aspects, the biomolecule is a protein comprising the FYS amino acid side chain (i.e., an “FSY protein”) of formula (II):

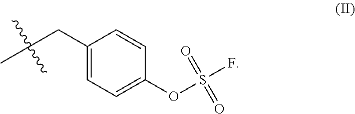 [SMILES: *Cc1ccc(OS(=O)(=O)F)cc1]

In embodiments, the protein comprises FSY that is proximal to serine, threonine, or a combination thereof. In aspects, the protein comprises FSY that is proximal to serine. In aspects, the protein comprises FSY that is proximal to threonine. In aspects, the protein comprises FSY that is proximal to serine and threonine. In aspects “proximal” means that FSY and serine and/or threonine are close enough to each other for a SuFEx reaction to successfully occur. In aspects, “proximal” means that FSY is within 1 to 20 amino acids of serine and/or threonine. In aspects “proximal” means that FSY is within 1 to 15 amino acids of serine and/or threonine. In aspects “proximal” means that FSY is within 1 to 10 amino acids of serine and/or threonine. In aspects “proximal” means that FSY is within 1 to 9 amino acids of serine and/or threonine. In aspects “proximal” means that FSY is within 1 to 8 amino acids of serine and/or threonine. In aspects “proximal” means that FSY is within 1 to 7 amino acids of serine and/or threonine. In aspects “proximal” means that FSY is within 1 to 6 amino acids of serine and/or threonine. In aspects “proximal” means that FSY is within 1 to 5 amino acids of serine and/or threonine. In aspects “proximal” means that FSY is within 1 to 4 amino acids of serine and/or threonine. In aspects “proximal” means that FSY is within 1 to 3 amino acids of serine and/or threonine. In aspects “proximal” means that FSY is within 1 to 2 amino acids of serine and/or threonine. In aspects “proximal” means that FSY is adjacent (next to) serine and/or threonine. In aspects, FSY and the serine and/or threonine are in a protein loop. In aspects, FSY and the serine and/or threonine are in a protein α-helix. In aspects, FSY and the serine and/or threonine are in a protein β-strand. In aspects, the disclosure provides a cell comprising the protein.

In embodiments, the protein comprises FSY (i.e., the “FSY protein”) that is proximal to dehydroalanine (Dha), dehydrobutyrine (Dhb), or a combination thereof. In aspects, the protein comprises FSY that is proximal to dehydroalanine. In aspects, the protein comprises FSY that is proximal to dehydrobutyrine. In aspects, the protein comprises FSY that is proximal to dehydroalanine and dehydrobutyrine. In aspects, “proximal” means that FSY is within 1 to 20 amino acids of dehydroalanine and/or dehydrobutyrine. In aspects “proximal” means that FSY is within 1 to 15 amino acids of dehydroalanine and/or dehydrobutyrine. In aspects “proximal” means that FSY is within 1 to 10 amino acids of dehydroalanine and/or dehydrobutyrine. In aspects “proximal” means that FSY is within 1 to 9 amino acids of dehydroalanine and/or dehydrobutyrine. In aspects “proximal” means that FSY is within 1 to 8 amino acids of dehydroalanine and/or dehydrobutyrine. In aspects “proximal” means that FSY is within 1 to 7 amino acids of dehydroalanine and/or dehydrobutyrine. In aspects “proximal” means that FSY is within 1 to 6 amino acids of dehydroalanine and/or dehydrobutyrine. In aspects “proximal” means that FSY is within 1 to 5 amino acids of dehydroalanine and/or dehydrobutyrine. In aspects “proximal” means that FSY is within 1 to 4 amino acids of dehydroalanine and/or dehydrobutyrine. In aspects “proximal” means that FSY is within 1 to 3 amino acids of dehydroalanine and/or dehydrobutyrine. In aspects “proximal” means that FSY is within 1 to 2 amino acids of dehydroalanine and/or dehydrobutyrine. In aspects “proximal” means that FSY is adjacent (next to) dehydroalanine and/or dehydrobutyrine. In aspects, FSY and the dehydroalanine and/or dehydrobutyrine are in a protein loop. In aspects, FSY and the dehydroalanine and/or dehydrobutyrine are in a protein α-helix. In aspects, FSY and the dehydroalanine and/or dehydrobutyrine are in a protein β-strand. In aspects, the disclosure provides a cell comprising the protein.

In embodiments, the protein comprises tyrosine that is proximal to dehydroalanine (Dha), dehydrobutyrine (Dhb), or a combination thereof. In aspects, the protein comprises tyrosine that is proximal to dehydroalanine. In aspects, the protein comprises tyrosine that is proximal to dehydrobutyrine. In aspects, the protein comprises tyrosine that is proximal to dehydroalanine and dehydrobutyrine. In aspects, “proximal” means that tyrosine is within 1 to 20 amino acids of dehydroalanine and/or dehydrobutyrine. In aspects “proximal” means that tyrosine is within 1 to 15 amino acids of dehydroalanine and/or dehydrobutyrine. In aspects “proximal” means that FSY is within 1 to 10 amino acids of dehydroalanine and/or dehydrobutyrine. In aspects “proximal” means that tyrosine is within 1 to 9 amino acids of dehydroalanine and/or dehydrobutyrine. In aspects “proximal” means that tyrosine is within 1 to 8 amino acids of dehydroalanine and/or dehydrobutyrine. In aspects “proximal” means that tyrosine is within 1 to 7 amino acids of dehydroalanine and/or dehydrobutyrine. In aspects “proximal” means that tyrosine is within 1 to 6 amino acids of dehydroalanine and/or dehydrobutyrine. In aspects “proximal” means that tyrosine is within 1 to 5 amino acids of dehydroalanine and/or dehydrobutyrine. In aspects “proximal” means that tyrosine is within 1 to 4 amino acids of dehydroalanine and/or dehydrobutyrine. In aspects “proximal” means that tyrosine is within 1 to 3 amino acids of dehydroalanine and/or dehydrobutyrine. In aspects “proximal” means that tyrosine is within 1 to 2 amino acids of dehydroalanine and/or dehydrobutyrine. In aspects “proximal” means that tyrosine is adjacent (next to) dehydroalanine and/or dehydrobutyrine. In aspects, tyrosine and the dehydroalanine and/or dehydrobutyrine are in a protein loop. In aspects, tyrosine and the dehydroalanine and/or dehydrobutyrine are in a protein α-helix. In aspects, tyrosine and the dehydroalanine and/or dehydrobutyrine are in a protein β-strand. In aspects, the disclosure provides a cell comprising the protein.

In embodiments, the disclosure provides protein complexes. In aspects, the protein complexes comprise two or more proteins. In aspects, the protein complexes comprise two proteins. In aspects, the protein complex comprises a first protein comprising fluorosulfate-L-tyrosine (i.e., the first protein is an “FSY protein”), and (ii) a second protein comprising serine, threonine, or a combination thereof; wherein the fluorosulfate-L-tyrosine in the first protein is proximal to the serine, threonine, or the combination thereof in the second protein. In aspects, the second protein comprises serine that is proximal to the fluorosulfate-L-tyrosine in the first protein. In aspects, the second protein comprises threonine that is proximal to the fluorosulfate-L-tyrosine in the first protein. In aspects, the second protein comprises serine and threonine that are proximal to the fluorosulfate-L-tyrosine in the first protein. In aspects, FSY and the serine and/or threonine are in a protein loop. In aspects, FSY and the serine and/or threonine are in a protein α-helix. In aspects, FSY and the serine and/or threonine are in a protein β-strand. In aspects, the disclosure provides a cell comprising the protein complex.

In aspects, the protein complex comprises a first protein comprising fluorosulfate-L-tyrosine (i.e., the first protein is an “FSY protein”), and (ii) a second protein comprising dehydroalanine, dehydrobutyrine, or a combination thereof; wherein the fluorosulfate-L-tyrosine in the first protein is proximal to the dehydroalanine, dehydrobutyrine, or the combination thereof in the second protein. In aspects, the second protein comprises dehydroalanine that is proximal to the fluorosulfate-L-tyrosine in the first protein. In aspects, the second protein comprises dehydrobutyrine that is proximal to the fluorosulfate-L-tyrosine in the first protein. In aspects, the second protein comprises dehydroalanine and dehydrobutyrine that are proximal to the fluorosulfate-L-tyrosine in the first protein. In aspects, FSY and the dehydroalanine and/or dehydrobutyrine are in a protein loop. In aspects, FSY and the dehydroalanine and/or dehydrobutyrine are in a protein α-helix. In aspects, FSY and the dehydroalanine and/or dehydrobutyrine are in a protein β-strand. In aspects, the disclosure provides a cell comprising the protein complex. In aspects, the proteins are proximal to each other but not bound together. In aspects, the proteins are covalently bonded together. In aspects, proteins are ionically bonded together. In aspects, the proteins are covalently and ionically bonded together.

In aspects, the protein complex comprises a first protein comprising tyrosine, and (ii) a second protein comprising dehydroalanine, dehydrobutyrine, or a combination thereof; wherein the tyrosine in the first protein is proximal to the dehydroalanine, dehydrobutyrine, or the combination thereof in the second protein. In aspects, the second protein comprises dehydroalanine that is proximal to the tyrosine in the first protein. In aspects, the second protein comprises dehydrobutyrine that is proximal to the tyrosine in the first protein. In aspects, the second protein comprises dehydroalanine and dehydrobutyrine that are proximal to the tyrosine in the first protein. In aspects, the tyrosine and the dehydroalanine and/or dehydrobutyrine are in a protein loop. In aspects, the tyrosine and the dehydroalanine and/or dehydrobutyrine are in a protein α-helix. In aspects, the tyrosine and the dehydroalanine and/or dehydrobutyrine are in a protein β-strand. In aspects, the disclosure provides a cell comprising the protein complex. In aspects, the proteins are proximal to each other but not bound together. In aspects, the proteins are covalently bonded together. In aspects, proteins are ionically bonded together. In aspects, the proteins are covalently and ionically bonded together.

Cellular Compositions

The disclosure provides cells comprising the compositions and complexes provided herein, including embodiments thereof. Therefore, in an aspect is provided a cell including fluorosulfate-L-tyrosine (FSY). In embodiments, the cell further includes a mutant pyrrolysyl-tRNA synthetase as described herein. In aspects, the cell further includes a vector as described herein. In aspects, the cell further includes a tRNA^{Pyl}.

In embodiments, FSY is biosynthesized inside the cell, thereby generating a cell containing FSY. In aspects, FSY is contained in the medium outside the cell and penetrates into the cell, thereby generating a cell containing FSY. In aspects, the cell comprises an FSY biomolecule. In aspects, the cell comprises an FSY protein. In aspects, the cell comprises an FSY biomolecule that is synthesized inside the cell. In aspects, the cell comprises an FSY protein that is synthesized inside the cell. In aspects, the cell comprises an FSY biomolecule that is synthesized outside a cell, and that penetrates into the cell. In aspects, the cell comprises an FSY protein that is synthesized outside a cell, and that penetrates into the cell.

A cell can be any prokaryotic or eukaryotic cell. For example, any of the compositions described herein can be expressed in bacterial cells such as E. coli, insect cells, yeast or mammalian cells (such as Hela cells, Chinese hamster ovary cells (CHO) or COS cells). In aspects, a cell can be a premature mammalian cell, i.e., pluripotent stem cell. In aspects, a cell can be derived from other human tissue. Other suitable cells are known to those skilled in the art.

Methods of Forming a Biomolecule

The compositions provided herein are useful for forming a biomolecule comprising an unnatural amino acid (e.g., FSY). Thus, in an aspect is provided method of forming an FSY biomolecule by contacting a biomolecule, a mutant pyrrolysyl-tRNA synthetase, a tRNA^{Pyl}, and fluorosulfate-L-tyrosine (FSY) having Formula (I):

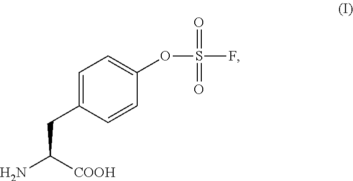 [SMILES: N[C@@H](Cc1ccc(OS(=O)(=O)F)cc1)C(=O)O]

thereby producing the FSY biomolecule, i.e., a biomolecule comprising the unnatural amino acid of FSY. The biomolecule produced by the method will comprise the unnatural amino acid side chain of Formula (II):

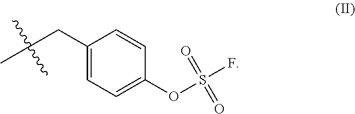 [SMILES: *Cc1ccc(OS(=O)(=O)F)cc1]

The mutant pyrrolysyl-tRNA synthetase used in the method of producing the biomolecule is any described herein. The tRNA^{Pyl} used in the method of producing the biomolecule is any described herein. In aspects, the biomolecule is a protein. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

In embodiments, the disclosure provides methods for producing an FSY protein by contacting a protein, a mutant pyrrolysyl-tRNA synthetase, a tRNA^{Pyl}, and fluorosulfate-L-tyrosine (FSY), thereby producing the FSY protein, i.e., a protein comprising the unnatural amino acid of FSY. The protein produced by the method will comprise the unnatural amino acid side chain of Formula (II):

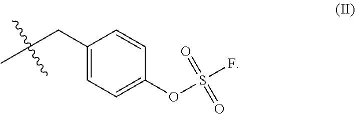 [SMILES: *Cc1ccc(OS(=O)(=O)F)cc1]

The mutant pyrrolysyl-tRNA synthetase used in the method of producing the protein is any described herein. The tRNA^{Pyl} used in the method of producing the protein is any described herein. In aspects, the FSY protein further comprises serine, threonine, or a combination thereof. In aspects, the FSY protein comprises FSY that is proximal to serine, threonine, or a combination thereof. In aspects, the FSY protein comprises FSY that is proximal to serine. In aspects, the FSY protein comprises FSY that is proximal to threonine. The term “proximal” is described herein. The FSY and serine and/or threonine that are proximal thereto can be on a protein loop. The FSY and serine and/or threonine that are proximal thereto can be on a protein α-helix and/or a protein β-strand. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

Forming Chemically Reactive Amino Acids

The disclosure provides methods of converting an amino acid to a chemically reactive amino acid, the method comprising contacting FSY with the amino acid; thereby converting the amino acid to a chemically reactive amino acid. In aspects, the method comprises contacting FSY with serine, threonine, or combination thereof, whereby the FSY converts the serine and/or threonine to dehydroalanine and/or dehydrobutyrine, respectively. In aspects, the method comprises contacting FSY with serine, whereby the FSY converts the serine to dehydroalanine. In aspects, the method comprises contacting FSY with threonine, whereby the FSY converts the threonine to dehydrobutyrine. In aspects, the method comprises contacting FSY with serine and threonine, whereby the FSY converts the serine to dehydroalanine, and converts the threonine to dehydrobutyrine. In aspects, FSY converts to tyrosine after converting the serine and/or threonine to dehydroalanine and/or dehydrobutyrine, respectively. In aspects, the FSY and the amino acid (e.g., serine and/or threonine) are in the same protein. In aspects, the FSY is in a first protein and the amino acid (e.g., serine and/or threonine) is in a second protein. In aspects, the method comprises contacting a first protein comprising FSY with a second protein comprising serine and/or threonine. In aspects, the reaction to form the chemically reactive amino acids (e.g., Dha, Dhb) is accomplished through click chemistry. In aspects, the reaction to form the chemically reactive amino acids (e.g., Dha, Dhb) is accomplished through proximity-enabled, click chemistry. In aspects, the reaction to form the chemically reactive amino acids (e.g., Dha, Dhb) is accomplished through a sulfur-fluoride exchange reaction. In aspects, the reaction to form the chemically reactive amino acids (e.g., Dha, Dhb) is accomplished through a proximity-enabled, sulfur-fluoride exchange reaction. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

The disclosure provides methods of converting an amino acid to a chemically reactive amino acid, the method comprising contacting an FSY protein with the amino acid; thereby converting the amino acid to a chemically reactive amino acid. In aspects, the method comprises contacting the FSY amino acid in the FSY protein with an amino acid in the FSY protein, whereby the FSY amino acid converts the amino acid in the FSY protein to a chemically reactive amino acid in the FSY protein. In aspects, the method comprises contacting the FSY amino acid in the FSY protein with serine, threonine, or combination thereof in the FSY protein, whereby the FSY amino acid converts the serine and/or threonine to dehydroalanine and/or dehydrobutyrine, respectively. In aspects, the method comprises contacting the FSY amino acid in the FSY protein with serine in the FSY protein, whereby the FSY amino acid converts the serine to dehydroalanine. In aspects, the method comprises contacting the FSY amino acid in the FSY protein with threonine in the FSY protein, whereby the FSY amino acid converts the threonine to dehydrobutyrine. In aspects, the method comprises contacting the FSY amino acid in the FSY protein with serine and threonine in the FSY protein, whereby the FSY amino acid converts the serine to dehydroalanine, and converts the threonine to dehydrobutyrine. In aspects, the FSY amino acid converts to tyrosine after converting the serine and/or threonine to dehydroalanine and/or dehydrobutyrine, respectively. In aspects, the reaction to form the chemically reactive amino acids is accomplished through click chemistry. In aspects, the reaction to form the chemically reactive amino acids is accomplished through proximity-enabled, click chemistry. In aspects, the reaction to form the chemically reactive amino acids is accomplished through a sulfur-fluoride exchange reaction. In aspects, the reaction to form the chemically reactive amino acids is accomplished through a proximity-enabled, sulfur-fluoride exchange reaction. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

The disclosure provides methods of converting an amino acid to a chemically reactive amino acid, the method comprising contacting an FSY protein with the amino acid in a second protein; thereby converting the amino acid in the second protein to a chemically reactive amino acid. In aspects, the method comprises contacting the FSY amino acid in the FSY protein with serine, threonine, or combination thereof in the second protein, whereby the FSY amino acid converts the serine and/or threonine in the second protein to dehydroalanine and/or dehydrobutyrine, respectively. In aspects, the method comprises contacting the FSY amino acid in the FSY protein with serine in the second protein, whereby the FSY amino acid converts the serine in the second protein to dehydroalanine. In aspects, the method comprises contacting the FSY amino acid in the FSY protein with threonine in the second protein, whereby the FSY amino acid converts the threonine in the second protein to dehydrobutyrine. In aspects, the method comprises contacting the FSY amino acid in the FSY protein with serine and threonine in the second protein, whereby the FSY amino acid converts the serine to dehydroalanine, and converts the threonine to dehydrobutyrine. In aspects, the FSY amino acid converts to tyrosine after converting the serine and/or threonine to dehydroalanine and/or dehydrobutyrine, respectively. In aspects, the reaction to form the chemically reactive amino acids is accomplished through click chemistry. In aspects, the reaction to form the chemically reactive amino acids is accomplished through proximity-enabled, click chemistry. In aspects, the reaction to form the chemically reactive amino acids is accomplished through a sulfur-fluoride exchange reaction. In aspects, the reaction to form the chemically reactive amino acids is accomplished through a proximity-enabled, sulfur-fluoride exchange reaction. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

Glycoprotein Mimetics

In embodiments, the disclosure provide methods of forming glycoprotein mimetics. In aspects, the method comprises: (i) contacting FSY in an FSY protein with an amino acid (e.g., serine and/or threonine) in the FSY protein, whereby the FSY amino acid converts the amino acid in the FSY protein to a chemically reactive amino acid (e.g., Dha and/or Dhb) in the FSY protein; and (ii) reacting the chemically reactive amino acid (e.g., Dha and/or Dhb) with a desired reactant to form a glycoprotein mimetic. In aspects, the method comprises: (i) contacting FSY in the FSY protein with serine, threonine, or combination thereof in the FSY protein, whereby FSY converts the serine and/or threonine to dehydroalanine and/or dehydrobutyrine, respectively; and (ii) reacting dehydroalanine and/or dehydrobutyrine with a desired reactant to form a glycoprotein mimetic. In aspects, the method comprises: (i) contacting FSY in the FSY protein with serine in the FSY protein, whereby the FSY amino acid converts the serine to dehydroalanine; and (ii) reacting dehydroalanine with a desired reactant to form a glycoprotein mimetic. In aspects, the method comprises: (i) contacting FSY in the FSY protein with threonine in the FSY protein, whereby the FSY amino acid converts the threonine to dehydrobutyrine; and (ii) reacting dehydrobutyrine with a desired reactant to form a glycoprotein mimetic. In aspects, the method comprises: (i) contacting FSY in the FSY protein with serine and threonine in the FSY protein, whereby the FSY amino acid converts the serine to dehydroalanine, and converts the threonine to dehydrobutyrine; and (ii) reacting dehydroalanine with a desired reactant to form a glycoprotein mimetic and/or reacting dehydrobutyrine with a desired reactant to form a glycoprotein mimetic. In aspects, the desired reactant is a carbohydrate. In aspects, the desired reactant is a carbohydrate comprising a thiol group. In aspects, the desired reactant is a saccharide. In aspects, the desired reactant is saccharide comprising a thiol group. In aspects, the desired reactant is a monosaccharide. In aspects, the desired reactant is monosaccharide comprising a thiol group.

In embodiments, the method comprises: (i) contacting an FSY protein with the amino acid in a second protein; thereby converting the amino acid in the second protein to a chemically reactive amino acid; and (ii) reacting the chemically reactive amino acid with a desired reactant to form a glycoprotein mimetic. In aspects, the method comprises: (i) contacting FSY in the FSY protein with serine, threonine, or combination thereof in a second protein, whereby FSY converts the serine and/or threonine in the second protein to dehydroalanine and/or dehydrobutyrine, respectively; and (ii) reacting dehydroalanine and/or dehydrobutyrine with a desired reactant to form a glycoprotein mimetic. In aspects, the method comprises: (i) contacting FSY in the FSY protein with serine in a second protein, whereby FSY converts the serine in the second protein to dehydroalanine; and (ii) reacting dehydroalanine with a desired reactant to form a glycoprotein mimetic. In aspects, the method comprises: (i) contacting FSY in the FSY protein with threonine in a second protein, whereby FSY converts the threonine in the second protein to dehydrobutyrine; and (ii) reacting dehydrobutyrine with a desired reactant to form a glycoprotein mimetic. In aspects, the method comprises: (i) contacting FSY in the FSY protein with serine and threonine in a second protein, whereby FSY converts the serine to dehydroalanine, and converts the threonine to dehydrobutyrine; and (ii) reacting dehydroalanine and dehydrobutyrine with a desired reactant to form a glycoprotein mimetic. In aspects, the desired reactant is a carbohydrate. In aspects, the desired reactant is a carbohydrate comprising a thiol group. In aspects, the desired reactant is a saccharide. In aspects, the desired reactant is saccharide comprising a thiol group. In aspects, the desired reactant is a monosaccharide. In aspects, the desired reactant is monosaccharide comprising a thiol group.

Pyrrolysyl-tRNA Synthetase

As described herein, an unnatural amino acid (e.g., FSY) may be inserted into or replace a naturally occurring amino acid in a biomolecule (e.g., protein). In order for the unnatural amino acid to be inserted or replace an amino acid in a biomolecule (e.g., protein), it must be capable of being incorporated during proteinogenesis. Thus, the unnatural amino acid must be present on a transfer RNA molecule (tRNA) such that it may be used in translation. Loading of amino acids occurs via an aminoacyl-tRNA synthetase, which is an enzyme that facilitates the attachment of appropriate amino acids to tRNA molecules. However, the attachment of unnatural amino acids to tRNA may not necessarily be accomplished by the naturally occurring aminoacyl-tRNA synthetase. Engineered aminoacyl-tRNA synthetases mutant pyrrolysyl-tRNA synthetase (PyIRS)) may be useful for attaching unnatural amino acids to tRNA. A PyIRS mutant library was generated. Compared to previously described PyIRS mutant library, the PyIRS mutant library generated herein was constructed using the new small-intelligent mutagenesis approach that allows a greater number of amino acid residues to be mutated simultaneously (e.g., 10 amino acid residues). Out of 2.76×10⁷ clones selected and screened in total, one PyIRS mutant (in 6 clones) was identified that is capable of attaching FSY.

The disclosure provides a mutant pyrrolysyl-tRNA synthetase, including at least 5 amino acid residues substitutions within the substrate-binding site of the mutant pyrrolysyl-tRNA synthetase. In aspects, the mutant pyrrolysyl-tRNA synthetase comprises at least 5 amino acid residues substitutions in the amino acid sequence of SEQ ID NO:3. In aspects, the substrate-binding site includes residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO:3. In aspects, the at least 5 amino acid residues substitutions are a substitution for alanine at position 302, a substitution for asparagine at position 346, a substitution for cysteine at position 348, a substitution for tyrosine at position 384, and a substitution for tryptophan at position 417 as set forth in the amino acid sequence of SEQ ID NO:3. In aspects, the at least 5 amino acid residues substitutions are isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO:3.

In embodiments, the mutant pyrrolysyl-tRNA synthetase has the amino acid sequence of SEQ ID NO:1. In aspects, the mutant pyrrolysyl-tRNA synthetase includes an amino acid sequence of SEQ ID NO:1. In aspects, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO:1. In aspects, the mutant pyrrolysyl-tRNA synthetase has an amino acid sequence that has at least 80% identity to SEQ ID NO:1. In aspects, the mutant pyrrolysyl-tRNA synthetase has an amino acid sequence that has at least 85% identity to SEQ ID NO:1. In aspects, the mutant pyrrolysyl-tRNA synthetase has an amino acid sequence that has at least 90% identity to SEQ ID NO:1. In aspects, the mutant pyrrolysyl-tRNA synthetase has an amino acid sequence that has at least 91% identity to SEQ ID NO:1. In aspects, the mutant pyrrolysyl-tRNA synthetase has an amino acid sequence that has at least 92% identity to SEQ ID NO:1. In aspects, the mutant pyrrolysyl-tRNA synthetase has an amino acid sequence that has at least 93% identity to SEQ ID NO:1. In aspects, the mutant pyrrolysyl-tRNA synthetase has an amino acid sequence that has at least 94% identity to SEQ ID NO:1. In aspects, the mutant pyrrolysyl-tRNA synthetase has an amino acid sequence that has at least 95% identity to SEQ ID NO:1. In aspects, the mutant pyrrolysyl-tRNA synthetase has an amino acid sequence that has at least 96% identity to SEQ ID NO:1. In aspects, the mutant pyrrolysyl-tRNA synthetase has an amino acid sequence that has at least 97% identity to SEQ ID NO:1. In aspects, the mutant pyrrolysyl-tRNA synthetase has an amino acid sequence that has at least 98% identity to SEQ ID NO:1. In aspects, the mutant pyrrolysyl-tRNA synthetase has an amino acid sequence that has at least 99% identity to SEQ ID NO:1.

In embodiments, the mutant pyrrolysyl-tRNA synthetase is encoded by the nucleic acid sequence of SEQ ID NO:2. In aspects, the mutant pyrrolysyl-tRNA synthetase is encoded by a nucleic acid sequence including the sequence of SEQ ID NO:2. In aspects, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO: 2. In aspects, the mutant pyrrolysyl-tRNA synthetase has an amino acid sequence that has at least 80% identity to SEQ ID NO:2. In aspects, the mutant pyrrolysyl-tRNA synthetase has an amino acid sequence that has at least 85% identity to SEQ ID NO:2. In aspects, the mutant pyrrolysyl-tRNA synthetase has an amino acid sequence that has at least 90% identity to SEQ ID NO:2. In aspects, the mutant pyrrolysyl-tRNA synthetase has an amino acid sequence that has at least 91% identity to SEQ ID NO:2. In aspects, the mutant pyrrolysyl-tRNA synthetase has an amino acid sequence that has at least 92% identity to SEQ ID NO:2. In aspects, the mutant pyrrolysyl-tRNA synthetase has an amino acid sequence that has at least 93% identity to SEQ ID NO:2. In aspects, the mutant pyrrolysyl-tRNA synthetase has an amino acid sequence that has at least 94% identity to SEQ ID NO:2. In aspects, the mutant pyrrolysyl-tRNA synthetase has an amino acid sequence that has at least 95% identity to SEQ ID NO:2. In aspects, the mutant pyrrolysyl-tRNA synthetase has an amino acid sequence that has at least 96% identity to SEQ ID NO:2. In aspects, the mutant pyrrolysyl-tRNA synthetase has an amino acid sequence that has at least 97% identity to SEQ ID NO:2. In aspects, the mutant pyrrolysyl-tRNA synthetase has an amino acid sequence that has at least 98% identity to SEQ ID NO:2. In aspects, the mutant pyrrolysyl-tRNA synthetase has an amino acid sequence that has at least 99% identity to SEQ ID NO:2.

Vectors

The compositions (e.g., mutant pyrrolysyl-tRNA synthetase, tRNA^{Pyl}) provided herein may be delivered to cells using methods well known in the art. Thus, in an aspect is provided a vector including a nucleic acid sequence encoding a mutant pyrrolysyl-tRNA synthetase as described herein, including embodiments thereof. In aspects, the vector comprises a nucleic acid sequence encoding a mutant pyrrolysyl-tRNA synthetase that comprises at least 5 amino acid residues substitutions within the substrate-binding site of the mutant pyrrolysyl-tRNA synthetase. In aspects, the vector further includes a nucleic acid sequence encoding tRNA^{Pyl}. In aspects, the vector comprises a nucleic acid sequence encoding a mutant pyrrolysyl-tRNA synthetase that comprises at least 5 amino acid residues substitutions in the amino acid sequence of SEQ ID NO:3. In aspects, the vector further includes a nucleic acid sequence encoding tRNA^{Pyl}. In aspects, the vector comprises a nucleic acid sequence encoding a mutant pyrrolysyl-tRNA synthetase that comprises amino acid substitutions of residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO:3. In aspects, the vector further includes a nucleic acid sequence encoding tRNA^{Pyl}. In aspects, the vector comprises a nucleic acid sequence encoding a mutant pyrrolysyl-tRNA synthetase that comprises amino acid substitutions of residues alanine at position 302, a substitution for asparagine at position 346, a substitution for cysteine at position 348, a substitution for tyrosine at position 384, and a substitution for tryptophan at position 417 as set forth in the amino acid sequence of SEQ ID NO:3. In aspects, the vector further includes a nucleic acid sequence encoding tRNA^{Pyl}. In aspects, the vector comprises a nucleic acid sequence encoding a mutant pyrrolysyl-tRNA synthetase that comprises amino acid substitutions of residues isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO:3. In aspects, the vector further includes a nucleic acid sequence encoding tRNA^{Pyl}.

In embodiments, the nucleic acid sequence encoding tRNA^{Pyl} is the sequence set forth in SEQ ID NO:4. In aspects, the nucleic acid sequence encoding tRNA^{Pyl} comprises the sequence set forth in SEQ ID NO:4. In aspects, the nucleic acid sequence encoding tRNA^{Pyl} has a sequence that has at least 80%, 85%, 90%, 91%, 92%, 93%, 94%, 95%, 96%, 97%, 98%, 99%, or 100% identity to SEQ ID NO:4. In aspects, the nucleic acid sequence encoding tRNA^{Pyl} has a sequence that has at least 80%, identity to SEQ ID NO:4. In aspects, the nucleic acid sequence encoding tRNA^{Pyl} has a sequence that has at least 85%, identity to SEQ ID NO:4. In aspects, the nucleic acid sequence encoding tRNA^{Pyl} has a sequence that has at least 90%, identity to SEQ ID NO:4. In aspects, the nucleic acid sequence encoding tRNA^{Pyl} has a sequence that has at least 91%, identity to SEQ ID NO:4. In aspects, the nucleic acid sequence encoding tRNA^{Pyl} has a sequence that has at least 92%, identity to SEQ ID NO:4. In aspects, the nucleic acid sequence encoding tRNA^{Pyl} has a sequence that has at least 93%, identity to SEQ ID NO:4. In aspects, the nucleic acid sequence encoding tRNA^{Pyl} has a sequence that has at least 94%, identity to SEQ ID NO:4. In aspects, the nucleic acid sequence encoding tRNA^{Pyl} has a sequence that has at least 95%, identity to SEQ ID NO: 4. In aspects, the nucleic acid sequence encoding tRNA^{Pyl} has a sequence that has at least 96%, identity to SEQ ID NO:4. In aspects, the nucleic acid sequence encoding tRNA^{Pyl} has a sequence that has at least 97%, identity to SEQ ID NO:4. In aspects, the nucleic acid sequence encoding tRNA^{Pyl} has a sequence that has at least 98%, identity to SEQ ID NO:4. In aspects, the nucleic acid sequence encoding tRNA^{Pyl} has a sequence that has at least 99%, identity to SEQ ID NO:4.

### Embodiments P1-P22

Embodiment P1. A method of converting an amino acid to a chemically reactive amino acid, the method comprising: (i) contacting an FSY protein with the amino acid; thereby converting the amino acid to a chemically reactive amino acid.

Embodiment P2. The method of claim 1, further comprising glycosylating the reactive amino acid.

Embodiment P3. The method of claim 1 or 2, wherein the amino acid is serine and the chemically reactive amino acid is dehydroalanine.

Embodiment P4. The method of any one of claims 1 to 3, wherein the amino acid is threonine and the chemically reactive amino acid is dehydrobutyrine.

Embodiment P5. The method of any one of claims 1 to 4, wherein contacting comprises a sulfur-fluoride exchange reaction.

Embodiment P6. The method of claim 5, wherein contacting comprises a proximity-enabled, sulfur-fluoride exchange reaction.

Embodiment P7. The method of any one of claims 1 to 6, wherein the FSY protein comprises the amino acid.

Embodiment P8. The method of claim 7, wherein the amino acid is proximal to the fluorosulfate-L-tyrosine in the FSY protein.

Embodiment P9. The method of any one of claims 1 to 6, wherein the method comprises contacting the FSY protein with a second protein comprising the amino acid.

Embodiment P10. The method of any one of claims 7 to 9, wherein the amino acid and the fluorosulfate-L-tyrosine in the FSY protein are in a protein α-helix.

Embodiment P11. The method of any one of claims 7 to 9, wherein the amino acid and the fluorosulfate-L-tyrosine in the FSY protein are in a protein β-strand.

Embodiment P12. The method of any one of claims 7 to 9, wherein the amino acid and the fluorosulfate-L-tyrosine in the FSY protein are in a protein loop.

Embodiment P13. The method of any one of claims 1 to 12, wherein the contacting is performed within a cell.

Embodiment P14. The method of claim 13, wherein the cell is a bacterial cell.

Embodiment P15. The method of claim 13, wherein the cell is a mammalian cell.

Embodiment P16. The method of any one of claims 1 to 15, further comprising, prior to the contacting in step (i), performing the step: (ii) contacting a protein, a pyrrolysyl-tRNA synthetase, a tRNA^{Pyl}, and a fluorosulfate-L-tyrosine, thereby forming the FSY protein.

Embodiment P17. A protein comprising: (i) fluorosulfate-L-tyrosine, and (ii) serine, threonine, or a combination thereof proximal to the fluorosulfate-L-tyrosine.

Embodiment P18. A protein comprising: (i) fluorosulfate-L-tyrosine, and (ii) dehydroalanine, dehydrobutyrine, or a combination thereof proximal to the fluorosulfate-L-tyrosine.

Embodiment P19. A protein comprising: (i) tyrosine, and (ii) dehydroalanine, dehydrobutyrine, or a combination thereof proximal to the tyrosine.

Embodiment P20. A protein complex comprising: (i) a first protein comprising fluorosulfate-L-tyrosine, and (ii) a second protein comprising serine, threonine, or a combination thereof; wherein the fluorosulfate-L-tyrosine in the first protein is proximal to the serine, threonine, or the combination thereof in the second protein.

Embodiment P21. A protein complex comprising: (i) a first protein comprising fluorosulfate-L-tyrosine, and (ii) a second protein comprising dehydroalanine, dehydrobutyrine, or a combination thereof; wherein the fluorosulfate-L-tyrosine in the first protein is proximal to the dehydroalanine, dehydrobutyrine, or the combination thereof in the second protein.

Embodiment P22. A protein complex comprising: (i) a first protein comprising tyrosine, and (ii) a second protein comprising dehydroalanine, dehydrobutyrine, or a combination thereof; wherein the tyrosine in the first protein is proximal to the dehydroalanine, dehydrobutyrine, or the combination thereof in the second protein.

### EXAMPLES

The following examples are intended to further illustrate certain embodiments of the disclosure. The examples are put forth so as to provide one of ordinary skill in the art and are not intended to limit its scope.

### Example 1

FSY was synthesized using the SO₂F₂/borax method (88% yield). Dong et al, Angew. Chem. Int. Ed. Engl, 53:9430-9448 (2014); Chen et al, Angew. Chem. Int. Ed. Engl., 55:1835-1838 (2016). To genetically encode FSY, the inventors developed a mutant pyrrolysyl-tRNA synthetase (PylRS) specific for FSY. A PylRS mutant library was generated by mutating residues Ala302, Leu305, Tyr306, Leu309, Ile322, Asn346, Cys348, Tyr384, Val401, and Trp417 of the Methanosarcina mazei PylRS using the small-intelligent mutagenesis approach, and subjected to selection as described. Lacey et al, ChemBioChem, 14:2100-2105 (2013); Wang et al, Angew. Chem. Int. Ed. Engl., 44:34-66 (2005); Takimoto et al, ACS Chem. Biol., 6:733-743 (2011). Six hits showing FSY-dependent phenotype were identified; they all converged on the same amino acid sequence (302I/346T/348I/384L/417K) which is referred to herein as FSYRS.

The incorporation specificity of FSY into proteins in E. coli was evaluated. The Zₛₚₐ affibody (Afb) gene containing a TAG codon at position 36 (Afb-36TAG) was co-expressed with the tRNA^{Pyl}/FSYRS pair in E. coli. In the absence of FSY, no full-length Afb was detected; when 1 mM FSY was added in growth media, full-length Afb36FSY was produced with a yield of 1.6 mg/L. The purified Afb36FSY was analyzed by electrospray ionization time-of-flight mass spectrometry (ESI-TOF MS). A peak observed at 7855.96 Da corresponds to intact Afb containing FSY at site 36 (Afb36FSY: expected 7856.69 Da). A peak measured at 7724.77 Da corresponds to Afb36FSY lacking the initiating Met (Afb36FSY-Met: expected 7725.50 Da). Two minor peaks observed at 7836.55 and 7705.16 Da correspond to Afb36FSY lacking F (expected 7836.69 Da) and Afb36FSY-Met lacking F (expected 7705.49 Da), respectively, suggesting slight F elimination during MS measurement. Notably, no peaks corresponding to Afb containing other amino acids at position 36 were observed. FSY was also incorporated at position 24 of the Z protein and analyzed with tandem MS. A series of b and y ions unambiguously indicated that FSY was incorporated at the TAG-specified position 24. The presence of 1 mM FSY did not affect E. coli growth, indicating no obvious cytotoxicity. These results indicated that the evolved tRNA^{Pyl}/FSYRS pair was able to incorporate FSY with high efficiency and specificity in E. coli.

FSY incorporation into proteins in mammalian cells was tested. HeLa-EGFP-182TAG reporter cells were transfected with plasmid pMP-FSYRS-3×tRNA, which expresses FSYRS and tRNA^{Pyl} genes. Wang et al, Nat. Neurosci., 10:1063-1072 (2007). Suppression of the 182TAG codon would produce full-length EGFP rendering cells fluorescent. After transfection, cells were incubated with FSY of various concentrations at 37° C. for 24 or 48 h followed by flow cytometry. Strong EGFP fluorescence was measured from cells only when FSY was added. The fluorescence intensity increased with FSY concentration and incubation time. As a positive control, p-azido-L-phenylalanine (AzF) was incorporated into reporter cells in parallel using plasmid pIre-Azi3, which is the most efficient Uaa incorporation system in mammalian cells in our hands. Coin et al, Cell, 155:1258-1269 (2013). FSY incorporation compared favorably with AzF, reaching 76% of the AzF level. Notably, while cellular toxicity is often an issue with bioreactive Uaas, no obvious toxicity of FSY to HeLa or 293T cells was observed, a valuable characteristic of FSY possibly due to the extremely low background reactivity of aryl fluorosulfate inside cells. Chen et al, J. Am. Chem. Soc., 138:7353-7364 (2016). These results were also confirmed by fluorescence confocal microscopy. In the presence of FSY, strong EGFP fluorescence was observed throughout the cells, and cell morphology remained normal. No fluorescence signal was detected when FSY was not added. These results demonstrate that FSY was incorporated into proteins in mammalian cells with high efficiency and specificity without causing detrimental effects.

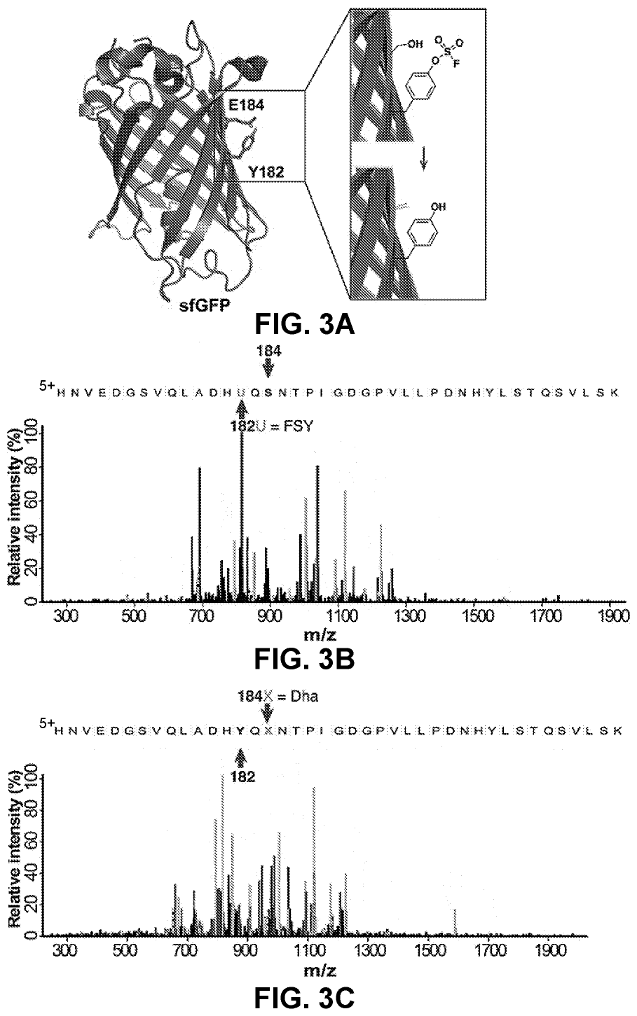

The inventors then determined whether the incorporated FSY could react with natural amino acid residues via proximity-enabled reactivity directly in E. coli. Afb binds to its substrate Z protein with a moderate affinity, providing a suitable protein framework to study FSY crosslinking in vivo. In light of the crystal structure of Afb-Z complex (Hogbom, et al, P. Proc. Natl. Acad. Sci. USA, 100:3191-3196 (2003)), the inventors introduced FSY at position 24 of Z protein and the target natural residue at position 7 of Afb, placing the two residues in close proximity upon Afb-Z binding (FIG. 3A). As aryl fluorosulfate is a weak electrophile, the inventors decided to test FSY's reactivity toward Lys, His, Tyr, Cys, Ser, and Thr using Ala as a negative control. To better separate the Afb and Z proteins of similar molecular weights, we fused maltose binding protein (MBP) to the N-terminus of Z (MBP-Z). MBP-Z and Afb were both appended with a 6×His-tag at C-terminus. To determine whether chemical crosslinking could occur in living cells, we co-expressed MBP-Z24FSY and Afb-7X (X=target residue) in E. coli. After culturing at 37° C. for 6 h, the same number of cells were analyzed using Western blot under denatured conditions. From cells expressing Afb-7Lys, Afb-7His, or Afb-7Tyr, crosslinking bands were observed with molecular weight corresponding to MBP-Z24FSY and Afb adducts. 6×His-tagged proteins were purified from cells and analyzed with SDS-PAGE. Consistently, a protein band corresponding to the crosslinked MBP-Z with Afb was clearly observed for Afb-7Lys, Afb-7His, and Afb-7Tyr, with crosslinking efficiency of 59%, 53% and 35%, respectively. In contrast, no cross-linking bands were observed when MBP-Z24FSY was co-expressed with Afb-7Cys, Afb-7Ser, Afb-7Thr, or Afb-7Ala. While aryl carbamate requires basic pH to crosslink Lys or Tyr at Afb/Z interface in vitro (Xuan et al, Angew. Chem. Int. Ed. Engl., 56:5096-5100 (2017)), FSY was able to crosslink Lys, His or Tyr directly in live E. coli cells, but did not crosslink Cys, Ser, or Thr.

To further validate the in vivo chemical crosslinking ability of FSY, the purified proteins were analyzed using tandem MS. As expected, strong signals corresponding to the covalently-linked peptides of MBP-Z24FSY and Afb-7Lys were identified (FIG. 3C). A series of b and y fragmented ions clearly indicated that the incorporated FSY crosslinked exclusively with Lys 7 of Afb. Similar MS results were also obtained for MBP-Z24FSY co-expressed with Afb-7His, confirming FSY crosslinked with the target His7. Meanwhile, consistent with Western and SDS-PAGE results, no crosslinked peptides of MBP-Z24FSY with Afb-7Ser, Afb-7Thr, Afb-7Cys, or Afb-7Ala were detected by tandem MS. Although crosslinking of MBP-Z24FSY with Afb-7Tyr was detected using Western and SDS-PAGE, the cross-linked peptides with tandem MS could not be identified.

Materials and Methods

Chemical synthesis of FSY: The fluorosulfate-L-tyrosine HCl salt was synthesized based on the classic SO₂F₂/borax method. Chen et al, Angew. Chem. Int. Ed. Engl. 2016, 55, 1835-1838; Dong et al, Angew. Chem. Int. Ed. Engl. 2014, 53, 9430-9448.

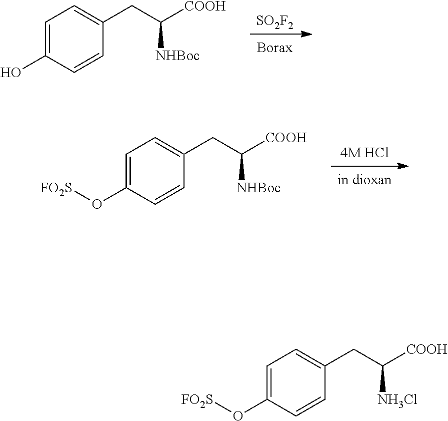

To a 2 L two-neck round-bottom flask containing a magnetic stir bar was added Boc-Tyr-OH (5.00 g, 17.8 mmol), 210 mL of CH₂Cl₂ and 860 mL of a saturated Borax solution. The mixture was stirred vigorously for 20 minutes. The reaction system was vacuumed until the biphasic solution started to degas and refilled with SO₂F₂ for three times. The reaction mixture was stirred vigorously at 25° C. overnight. CH₂Cl₂ was carefully removed using a rotary evaporator. Then 1 M aqueous HCl (210 mL) was slowly added to the reaction mixture while stirring and white solid precipitated. The mixture was filtered and the solid was washed with water (80 mL×3). The white solid was dried under vacuum (1 mm Hg) at 40° C. for 4 h affording 6.07 g (16.7 mmol) of the Boc-Tyr-OSO₂F, which was directly used in the next step without any further purification. Boc-Tyr-OSO₂F (2.0 g, 5.5 mmol) was treated with 4 M HCl in dioxane (11 mL) and the reaction mixture was stirred overnight, during which white solid precipitated. The solid was filtered and washed by cool ether (5 mL×2), affording the targeted fluorosulfate-L-tyrosine HCl salt as a white solid (1.46 g, 88% yield). ¹H NMR (400 MHz, CD₃OD): δ (ppm) 3.23-3.41 (m, 2H), 4.32-4.34 (m, 1H), 7.45-7.53 (m, 4H); ¹³C NMR (400 MHz, CD₃OD): δ (ppm) 38.9, 57.2, 125.0, 135.3, 139.5, 153.5, 173.3; MS: 264.0 [NH₃-Tyr-OSO₂F]⁺, 286.0 [NH₂-Tyr-OSO₂F+Na]⁺

Synthetase library construction and selection: The pBK-TK3 mutant library of MmPylRS was constructed using the new small-intelligent mutagenesis approach, which uses a single codon for each amino acid and thus allows a greater number of residues to be mutated simultaneously. The following residues of MmPylRS were mutated using the procedures previously described by Lacey et al, ChemBioChem, 14:2100-2105 (2013): 302NYT, 305WTG, 306WTG/TAC, 309KYA, 322AYA, 346NDT/VMA/ATG/TGG, 348NDT/VMA/ATG/TGG, 384TTM/TAT, 401VTT, 417NDT/VMA/ATG/TGG.

DH10B cells (100 uL) harboring the pREP positive selection reporter was transformed with 100 ng of pBK-TK3 library via electroporation. The electroporated cells were immediately recovered with 1 mL of pre-warmed SOC media and agitated vigorously at 37° C. for 1 h. The recovered cells were directly plated on a LB-agar selection plate supplemented with 1 mM FSY, 12.5 μg mL⁻¹ of tetracycline (Tet), 25 μg mL⁻¹ of kanamycin (Kan), and 68 μg mL⁻¹ of chloramphenicol (Cm). The selection plate was incubated at 37° C. for 48 h and then stored at room temperature. Colonies showing green fluorescence were diluted in 100 uL of LB and replicated on LB-agar screening plates containing 1) Tet12.5Kan25; 2) Tet12.5Kan25Cm100; 3) Tet12.5Kan25Cm100 supplemented with 1 mM FSY. After 48 h of incubation at 37° C., 6 clones present FSY-dependent fluorescence and growth were considered as hits and further characterized. The pBK plasmids encoding PylRS mutants were extracted by miniprep and then separated from reporter plasmids by DNA gel electrophoresis. The purified pBK plasmids were analyzed by Sanger-sequencing.

Plasmid Construction

pEvol-FSY: pEvol-FSY plasmid was generated by introducing the FSYRS encoding gene into pEvol vector via ligation independent cloning. Li et al, S. J. Nat. Methods, 4:251-256 (2007). Briefly, the FSYRS gene was amplified with following primers, purified, and ligated into pEvol vectors (linearized with Bgl II and Sal I) with T4 DNA polymerase. FSRYS-BglII-F is SEQ ID NO:5. FSYRS-SalI-R is SEQ ID NO:6.

pMP-3×tRNA^{Pyl}-FSYRS: The pMP-3×tRNA^{Py}-FSYRS plasmid was constructed by introducing the FSYRS gene into pMP vector via standard cloning. The FSYRS gene was amplified with following primers, digested with Nco I and Nhe I, and ligated into the pMP vector pre-treated with the same restriction enzymes. FSYRS-NcoI-F is SEQ ID NO:7. FSYRS-NheI-R is SEQ ID NO:8.

pET-Duet-Afb_{4A}-7X-MBP-Z24TAG: To evaluate the in vivo crosslinking ability of FSY, pET-Duet-Afb_{4A}-7X-MBP-Z24TAG plasmids were generated by introducing mutations at residue 7 of Afb_{4A}-7X (X=Lys, Tyr, Cys, Ser, Thr, His, or Ala) gene within the pET-Duet-MBP-Z24TAG expression vector via site-directed mutagenesis. Yang et al, Nat. Communi, 8:2240 (2017). The following primers were used. Afb-4A7A-F is SEQ ID NO:9. Afb-4A7K-F is SEQ ID NO:10.

pTak-CaM-76TAG-80Tyr: To investigate the intramolecular crosslinking ability of FSY, residue 76 and 80 of calmodulin encoding gene CaM were mutated to an amber stop codon TAG and Tyr respectively. Meanwhile, residue 75, 77, 79, 81 of CaM were mutated to Ala via overlapping PCR to assist the crosslinking reaction. The CaM gene was amplified with following primers, digested with Spe I and Blp I, and ligated into the pTak-CaM vector pre-treated with the same restriction enzymes. CaM-SpeI-F is SEQ ID NO:18. 80Tyr-R is SEQ ID NO:19. 80Tyr-F is SEQ ID NO:20.

pBad-CysH: To generate pBad-CysH plasmid, the PAPS reductase encoding gene CysH was amplified by colony PCR, digested with Nde I and Hind III, and ligated into the pBad vector pre-treated with the same restriction enzymes. CysH-NdeI-F is SEQ ID NO:22. CysH-Hind3-R is SEQ ID NO:23.

pBad-Trx35A62TAG: To generate pBad-Trx35A62TAG plasmid, residue 62 of Trx35A gene was mutated into an amber stop codon TAG using site-directed mutagenesis with following primers. Trx-62TAG-F is SEQ ID NO:24. Trx-62TAG-R is SEQ ID NO:25.

Protein Expression:

Afb36FSY: pTak-Afb36TAG-His and pBK-FSYRS were co-transformed into DH10B E. coli chemical competent cells. The transformants were plated on an LB-Kan50Cm34 agar plate and incubated overnight at 37° C. A single colony was inoculated into 5 mL of 2×YT-Kan50Cm34 and cultured overnight at 37° C. On the following day, 2 mL of overnight cell culture was diluted into 100 mL 2×YT-Kan50Cm34 and agitated vigorously at 37° C. When OD₆₀₀ reached 0.4˜0.6, half of the cell culture (50 mL) was supplemented with 1 mM FSY and 0.5 mM IPTG, then induced at 30° C. for 6 h. As a negative control, the rest 50 mL cell culture was induced with 0.5 mM IPTG at 30° C. for 6 h. Cell pellets were collected by centrifugation at 4200 g for 30 min at 4° C. and stored at −80° C.

Afb_{4A}-7X and MBP-Z24FSY: The pEvol-FSYRS and pET-Duet-Afb_{4A}-7X-MBP-Z24TAG were co-transformed into BL21(DE3) E. coli chemical competent cells. The transformants were plated on an LB-Amp100Cm34 agar plate and incubated overnight at 37° C. A single colony was inoculated into 5 mL of 2×YT-Amp100Cm34 and cultured overnight at 37° C. On the following day, 1 mL of overnight cell culture was diluted into 50 mL 2×YT-Amp100Cm34 and agitated vigorously at 37° C. When OD₆₀₀ reached 0.4˜0.6, the cell culture was induced with 0.5 mM IPTG and 0.2% arabinose, then incubated at 37° C. for 6 h. Cell pellets were collected by centrifugation at 4200 g for 30 min at 4° C. and stored at −80° C.

CaM-76FSY-80Tyr: pBad-CaM76TAG80Tyr and pEvol-FSYRS were co-transformed into BL21(DE3) E. coli chemical competent cells. The transformants were plated on an LB-Amp100Cm34 agar plate and incubated overnight at 37° C. A single colony was inoculated into 5 mL of 2×YT-Amp100Cm34 and cultured overnight at 37° C. On the following day, 1 mL of overnight cell culture was diluted into 50 mL 2×YT-Amp100Cm34 and agitated vigorously at 37° C. When OD₆₀₀ reached 0.4˜0.6, the cell culture was induced with 0.2% arabinose, then incubated at 37° C. for 6 h. Cell pellets were collected by centrifugation at 4200 g for 30 min at 4° C. and stored at −80° C.

Trx35A62FSY: pBad-Trx35A62TAG and pEvol-FSYRS were co-transformed into BL21(DE3) E. coli chemical competent cells. The transformants were plated on an LB-Amp100Cm34 agar plate and incubated overnight at 37° C. A single colony was inoculated into 5 mL of 2×YT-Amp100Cm34 and cultured overnight at 37° C. On the following day, 1 mL of overnight cell culture was diluted into 50 mL 2×YT-Amp100Cm34 and agitated vigorously at 37° C. When OD₆₀₀ reached 0.4˜0.6, the cell culture was induced with 0.2% arabinose, then incubated at 30° C. for 6 h. Cell pellets were collected by centrifugation at 4200 g for 30 min at 4° C. and stored at −80° C.

PAPS reductase: pBad-CysH was transformed into DH10B E. coli chemical competent cells. The transformants were plated on an LB-Amp100 agar plate and incubated overnight at 37° C. A single colony was inoculated into 10 mL of 2×YT-Amp100 and cultured overnight at 37° C. On the following day, 10 mL of overnight cell culture was diluted into 1 L 2×YT-Amp100 and agitated vigorously at 37° C. When OD₆₀₀ reached 0.4˜0.6, the cell culture was induced with 0.2% arabinose, then incubated at 30° C. for 6 h. Cell pellets were collected by centrifugation at 4200 g for 30 min at 4° C. and stored at −80° C.

His-tag protein purification: Above cell pellets were resuspended in 14 mL lysis buffer (50 mM Tris-HCl pH 8.0, 500 mM NaCl, 20 mM imidazole, 1% v/v Tween 20, 10% v/v glycerol, lysozyme 1 mg/mL, DNase 0.1 mg/mL, and protease inhibitors). The cell suspension was lysed at 4° C. for 30 min. Cell lysate was sonicated with Sonic Dismembrator (Fisher Scientific, 30% output, 3 min, 1 sec off, 1 sec on) in an ice-water bath, followed by centrifugation (20,000 g, 30 min, 4° C.). The soluble fractions were collected and incubated with pre-equilibrated Protino®Ni-NTA Agarose resin (400 μL) at 4° C. for 1 h with constant mechanical rotation. The slurry was loaded onto a Poly-Prep® Chromatography Column, washed with 5 mL of wash buffer (50 mM Tris-HCl pH 8.0, 500 mM NaCl, 20 mM imidazole, and 10% v/v glycerol) for 3 times, and eluted with 200 μL of elution buffer (50 mM Tris-HCl, pH 8.0, 500 mM NaCl, 250 mM imidazole, and 10% v/v glycerol) for 5 times. The eluates were concentrated and buffer exchanged into 100 μL of protein storage buffer (50 mM Tris-HCl, pH 7.4 or 8.0, and 150 mM NaCl) using Amicon Ultra columns, and stored at −80° C. for future analysis.

FACS analysis of Uaa incorporation into HeLa-GFP-182TAG reporter cells: One day before transfection, 4.5×10⁴ HeLa-EGFP-182TAG reporter cells (Wang et al, Nat. Neurosci., 10:1063-1072 (2007)) were seeded in a Greiner bio-one 24 well-cell culture dish containing 500 μL of DMEM media with 10% FBS, and incubated at 37° C. in a CO₂ incubator. Plasmid pMP-3×tRNA-FSYRS (500 ng, encoding FSYRS and 3 copies of tRNA^{Pyl}) was transfected into target cells using 2.5 μL of lipofectamine 2000 following manufacturer's instructions. Six hours post transfection, the media containing transfection complex were replaced with fresh DMEM media with 10% FBS in the presence or absence of 1 mM FSY. For AzF incorporation, plasmid pIre-Azi3 (Coin et al, Cell, 155:1258-1269 (2013)) was similarly transfected and the DMEM media containing 10% FBS with or without 1 mM AzF were used. After incubation at 37° C. for 24-48 h, transfected cells were trypsinized and collected by centrifugation (1500 rpm, 5 min, r.t.). The cells were resuspended in 300 μL of FACS buffer (1×PBS, 2% FBS, 1 mM EDTA, 0.1% sodium azide, 0.28 μM DAPI) and analyzed by BD LSRFortessa™ cell analyzer.

Fluorescence confocal microscopy of HeLa-EGFP-182TAG reporter cells: One day before transfection, 4.5×10⁴ HeLa-EGFP-182TAG cells were seeded in a Greiner bio-one CELLview glass bottom dish containing 500 μL of DMEM media with 10% FBS, and incubated at 37° C. in a CO₂ incubator. Plasmid pMP-3×tRNA-FSYRS (500 ng) was transfected into target cells using 2.5 μL of lipofectamine 2000 following manufacturer's instructions. Six hours post transfection, the media were replaced with complete DMEM media with or without 1 mM FSY. The cells were incubated at 37° C. for additional 24-48 h and imaged with Nikon Eclipse Ti confocal microscope.

Mass spectrometric analysis: Intact FSY-containing Afb were analyzed by ESI-TOF MS using an Agilent 6210 mass spectrometer coupled to an Agilent 1100 HPLC system. Two micrograms of protein samples were injected by an auto-sampler and separated on an Agilent Zorbax SB-C8 column (2.1 mm ID×10 cm length) by a reverse-phase gradient of 0-80% acetonitrile for 15 min. Mass calibration was performed right before the analysis. Protein spectra were averaged and the charge states were deconvoluted using Agilent MassHunter software.

Protein digestion and tandem mass spectrometry measurement were performed as previously described by Yang et al, Nat. Communi., 8:2240 (2017). The Afb/MBP-Z samples were digested with Glu-C. The CaM and Trx1/PAPS reductase samples were digested by trypsin. Digested peptides were analyzed with an in-line EASY-spray source and nano-LC UltiMate 3000 high-performance liquid chromatography system (Thermo Fisher) interfaced with Elite mass spectrometer (Thermo Fisher). Peptides were eluted over gradient of 2%-40% buffer B (80% acetonitrile, 20% H₂O, 0.1% formic acid) at flow rate 300 nL/min from EASY-Spray PepMap C18 Columns (50 cm; particle size, 2 μm; pore size, 100 Å; Thermo Fisher). For different samples, slight modifications were made to the separation method. The Elite mass spectrometer was operated in data-dependent mode with one full MS scan at R=60,000 (m/z=200) mass range from 375 to 1800 (AGC target 1×10⁶), followed by ten CID MS/MS scans. A dynamic exclusion time of 30 s was used, and singly charged ions were excluded. Mass spectrometry raw data was searched by Maxquant.

### Example 2

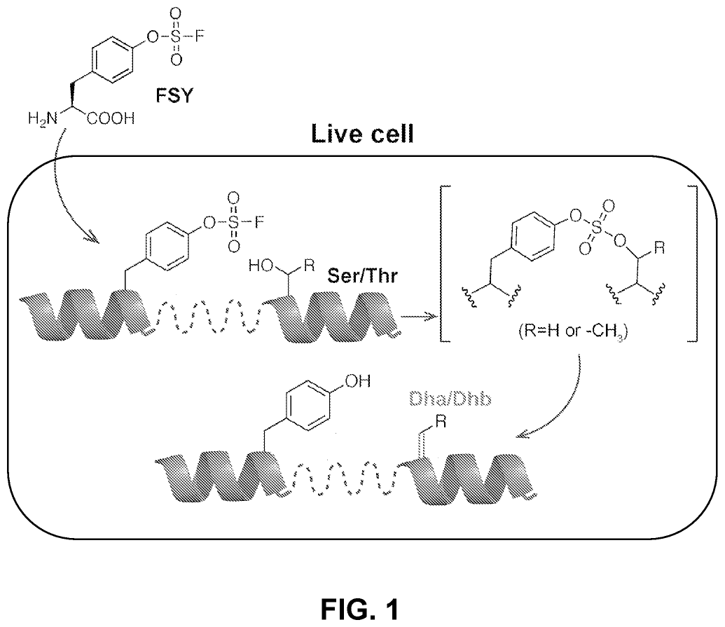

Here the inventors present a novel strategy to selectively introduce chemically reactive unnatural amino acids into proteins directly in live cells. The inventors genetically encoded a latent bioreactive unnatural amino acid (Uaa) into proteins at a site proximal to the target position; enabled by proximity-enhanced reactivity (Wang, N. Biotechnol., 2017, 38(Pt A):16-25 (2017)), the latent bioreactive Uaa then reacted with the nearby target natural amino acid residue, selectively converting it into a chemically reactive Uaa (FIG. 1). Through incorporating the latent bioreactive Uaa fluorosulfate-L-tyrosine (FSY) and harnessing its reaction with Ser or Thr, the inventors demonstrated the in vivo generation of Dha and Dhb on proteins in E. coli cells. This strategy worked on various proteins and secondary structures. The inventors further showed that the resultant Dha could be used to selectively attach saccharide to proteins for generating glycoprotein mimetics. The inventors expect that Dha and Dhb generated in vivo will enable versatile chemical means for protein research and engineering in live cells, and that this Genetically Encoded Chemical Conversion (GECCO) strategy will open a new avenue for selective introduction of chemically reactive amino acids in vivo.

As described in Example 1 and Wang et al, J. Am. Chem. Soc., 140:4995-4999 (2018), the inventors developed an orthogonal tRNA^{Pyl}/FSYRS pair that genetically incorporated Uaa FSY into proteins in E. coli and mammalian cells. The incorporated FSY was found to react with Lys, His, and Tyr in proximity through sulfur-fluoride exchange (SuFEx) reaction, forming covalent protein crosslinks in vivo. However, crosslinking was not detected between FSY and Ser or Thr. In contrast, arylfluorosulfate installed on chemical probes was able to react with Lys, Tyr, and Ser within a positively charged binding pocket of the specifically bound protein, and the resultant arylfluorosulfate-Ser adduct was found to partially hydrolyze to Dha, but what occurred to the arylfluorosulfate warhead remained uncharacterized. See Chen et al, J. Am. Chem. Soc. 2016, 138(23):7353-7364 (2016); Mortenson et al, J. Am. Chem. Soc. 2018, 140(1):200-210 (2018); Fadeyi et al, ACS Chem. Biol., 12(8):2015-2020 (2017). Prompted by these findings, the inventors determined whether proximal FSY and Ser incorporated into proteins (instead of on small molecules) would react, whether a positively charged microenvironment was necessary, and what the products would be. FSY and Ser were incorporated at different protein contexts without positively charged residues nearby, and their identity was characterized using high resolution tandem MS.

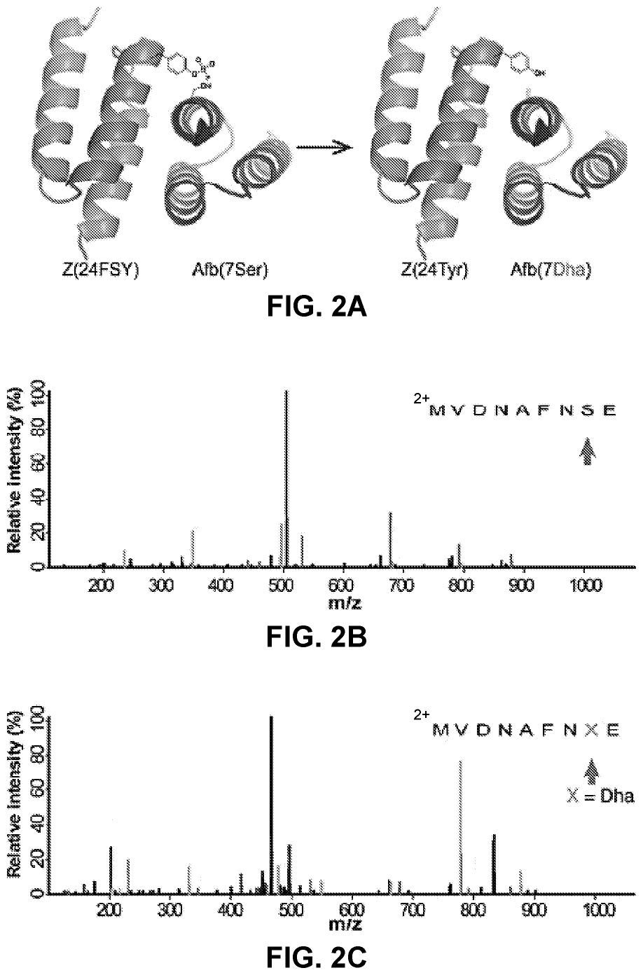

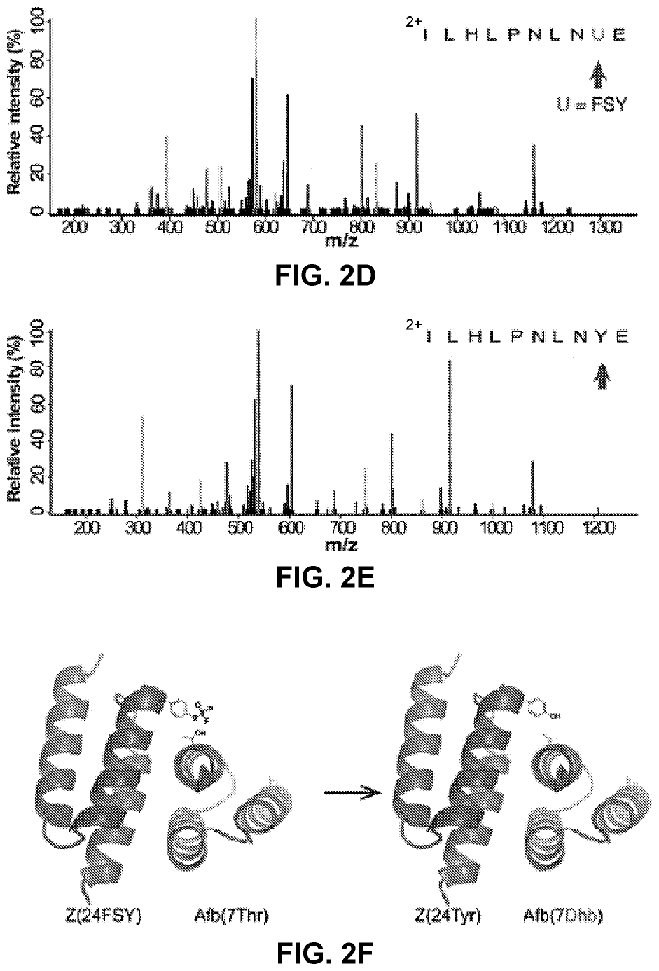

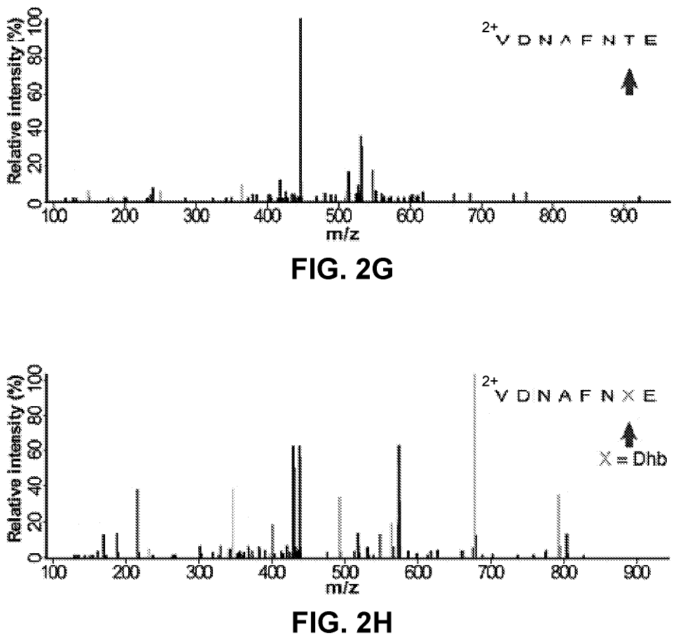

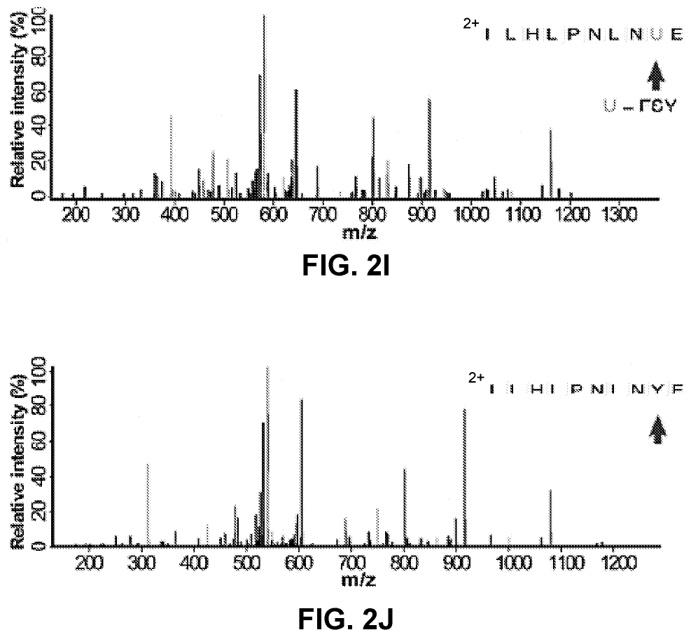

It was first examined whether FSY would react with Ser intermolecularly when separately installed on two interacting proteins. Specifically, FSY was incorporated at position E24 of the Z protein, and introduced Ser at position K7 of the Z-binding affibody (Afb) (FIG. 2A), placing them in proximity when Afb binds with Z. See Hogbom et al, Proc. Natl. Acad. Sci. USA, 100(6):3191-3196 (2003). Proteins Z(24FSY) and Afb(7Ser) were co-expressed in E. coli, allowing them to bind and react in vivo, and then purified and characterized using MS. Tandem MS identified the Ser-containing peptide for the Afb(7Ser) protein (FIG. 2B). This peptide with Dha at the 7Ser position (FIG. 2C) was also identified. A series of b and y ions in the tandem MS unambiguously indicated the presence of Dha at the 7Ser position, confirming the conversion of Ser to Dha. In addition, the FSY-containing peptide for the Z(24FSY) protein (FIG. 2D) was identified, indicating specific incorporation of FSY. The peptide containing Tyr at the 24FSY position was also identified (FIG. 2E), indicating the conversion of FSY to Tyr upon reacting with Ser. Based on the peak areas of these peptides in the extracted ion chromatograms, the conversion rate of Ser to Dha was ca. 3.7% and FSY to Tyr was ca. 4.1%. These results indicate that FSY installed proximal to Ser in proteins can intermolecularly convert Ser into Dha in vivo.

It was next examined whether FSY could convert Thr to Dhb in proteins in vivo. Similarly, proteins Z(24FSY) and Afb(7Thr) were co-expressed in E. coli (FIG. 2F). MS analyses of the purified proteins identified the Thr-containing peptide of Afb(7Thr) (FIG. 2G), and the Dhb-containing peptide was also identified (FIG. 2H), validating the conversion of 7Thr to Dhb. Again, for protein Z(24FSY), the FSY-containing peptide was identified (FIG. 2I), together with this peptide wherein FSY was converted into Tyr (FIG. 2J). Extracted ion chromatograms of these peptides indicate that the conversion rate of Thr to Dhb was ca. 5.5% and FSY to Tyr was ca. 7.2%. These data indicate that FSY was able to convert both Ser and Thr into the respective Dha and Dhb, while changing itself back to the natural amino acid Tyr.

It was then determined whether Dha could be generated by FSY through intramolecular conversion. A Ser located on a n-strand of the super-fold green fluorescent protein (sfGFP) was targeted. FSY was incorporated at the permissive site Tyr182 and a Ser was placed at site 184, the i+2 position so that both side chains pointing to the same side of the β-strand (FIG. 3A). This mutant sfGFP(182FSY/184Ser) was expressed in E. coli and then characterized by MS. The peptide containing FSY at site 182 and Ser at site 184 was identified, again indicating specific incorporation of FSY (FIG. 3B). As expected, this peptide was also identified containing Tyr at site 182 and Dha at site 184 (FIG. 3C). The conversion rate of Ser to Dha was 1.3%. No peptide containing FSY182/Dha184 was detected, that is, whenever Dha was detected at site 184, site 182 was always found to be Tyr, indicating that conversion of Ser to Dha was consequentially associated with conversion of FSY to tyrosine. The Dha conversion rate was low possibly because the rigidity of the sfGFP n-strand does not allow ample contact of FSY with Ser; computational modeling indicates that the side chains of FSY and Ser point away from each other in their sterically allowed rotamers.

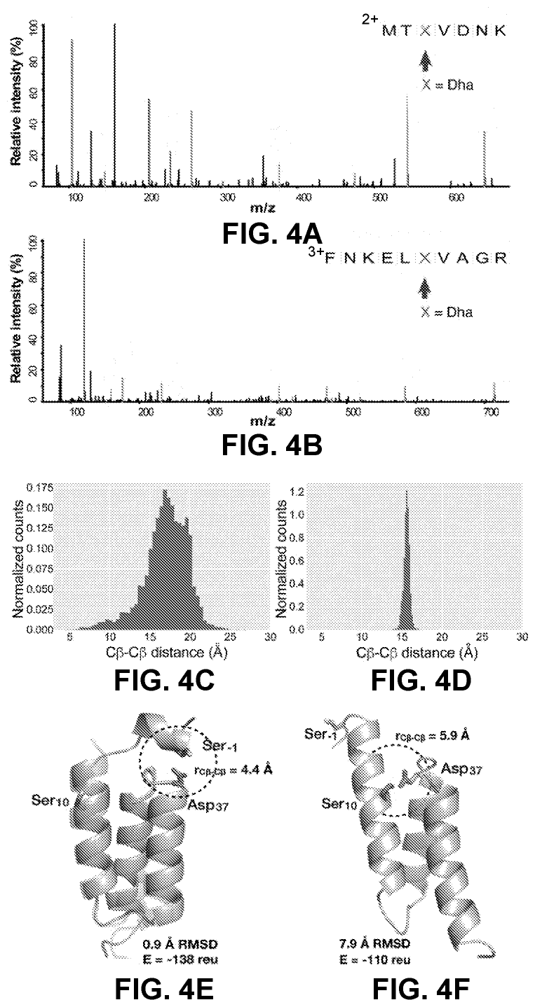

This intramolecular conversion was tested in a less rigid protein context in ubiquitin. FSY was introduced in site 45, which has contact with Ser65 and Lys63. After expressing ubiquitin(FSY45) in E. coli followed by MS characterization, FSY predominately crosslinked with Lys63 and Ser65 was converted to Dha in 1.7% yield. It was reasoned that a good contact of FSY with the target Ser without competition from Lys, His, and Tyr, which are known to react with FSY, would increase the conversion rate. FSY was incorporated into Afb at site Asp37, which is located in a loop and has no contact with Lys, His or Tyr, and it was determined how efficiently Ser could be converted into Dha. After expressing Afb(37FSY) in E. coli followed by purification, MS characterization revealed that Ser10 was converted to Dha in 3.9% yield and Ser-1 to Dha in 53% yield (FIGS. 4A-4B). The crystal structure of Afb is available only in complex with the Z protein, and some residues of Afb are missing in this complex structure. To understand the conversion difference, the inventors performed ab initio folding of the Afb sequence containing all residues, including additional residues Met(-3)Thr(-2)Ser(-1) at the N-terminus introduced by cloning. The C_{β}-C_{β} distances between site 37 and Ser-1 or Ser10 were analyzed in outputted models (FIGS. 4C-4D). Many low energy/low RMSD models contain Ser-1 with a close distance to site 37, whereas very few models contain Ser10 with a close distance to site 37—and these few models are very high energy (FIGS. 4C-4F). These data support that better contact of FSY with Ser-1 indeed enhances the conversion of Ser to Dha.

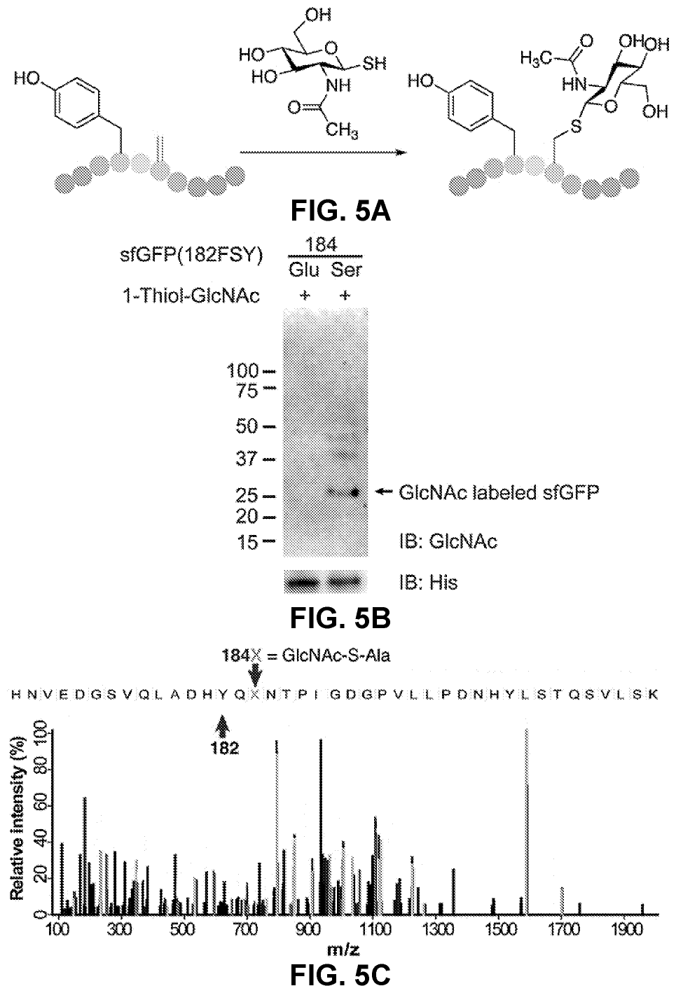

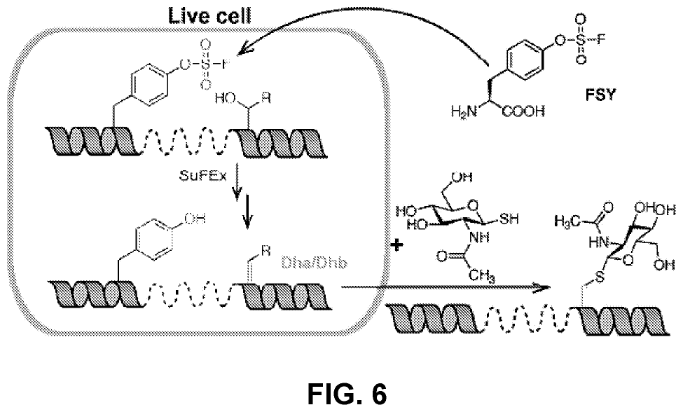

To further demonstrate the chemical identity of Dha generated by FSY conversion, Dha was labeled with a thiol-derivatized saccharide to generate glycoprotein mimetics (FIG. 5A, FIG. 6). Methods for preparing site-selectively glycosylated proteins and mimetics are valuable for studying protein glycosylation. See Wright et al, Science, 354(6312):aag1465-aag1465 (2016); Liu et al, J. Am. Chem. Soc., 125(7):1702-1703 (2003); Kiessling et al, Chem. Soc. Rev., 42(10):4476-4491 (2013); Tiwari et al, Chem. Rev., 116(5):3086-3240 (2016); Li et al, Chem. Rev., 118(17):8359-8413 (2018).

2-acetamido-2-deoxy-1-thio-β-D-glucopyranose (1-thiol-GlcNAc) was synthesized in 3 steps (68% overall yield), and incubated 1-thiol-GlcNAc with sfGFP(182FSY/184Ser) (expressed from E. coli as described above and containing 182Tyr/184Dha) under mild conditions. A similarly expressed and purified sfGFP(182FSY/184Glu) protein was used as the negative control. Western blot analysis of reaction product using an antibody specific for GlcNAc showed that only the Dha-containing sfGFP was labeled by 1-thiol-GlcNAc (FIG. 5B). The reaction product was further analyzed with tandem MS, which clearly confirmed the attachment of 1-thiol-GlcNAc onto Dha at site 184 (FIG. 5C). These results further validate the chemical identity of Dha generated by FSY conversion and its value for selective protein modification. See Dadová et al, Curr. Opin. Chem. Biol., 46:71-81 (2018).

In summary, the inventors developed a new method, GECCO, which enables genetically introducing biochemically reactive amino acids into proteins. Harnessing proximity-enabled reactivity, a genetically incorporated latent bioreactive Uaa converts a nearby target natural residue into a reactive amino acid in situ. The conversion of Ser and Thr into the reactive Dha and Dhb, respectively, has been demonstrated. In addition, the labeling of the Dha-containing protein with a thiol-saccharide to generate glycoprotein mimetics has been demonstrated. The conversion occurred both inter- and intra-molecularly on various proteins, with the conversion rate dependent on the contact between FSY and the target Ser/Thr. Dha and Dhb also represent two smallest Uaas introduced into proteins via genetic code expansion to date. Compared with existing methods for introducing Dha to proteins via chemical transformation (See Dadová et al, Curr. Opin. Chem. Biol., 46:71-81 (2018)), the disclosure provides a recombinant approach to produce Dha/Dhb-containing proteins without extra chemical treatments. The methods described herein will enable the genetic introduction of additional biochemically reactive amino acids in proteins, thus expanding new avenues for exploiting chemistry in live systems for biological research and engineering.

Materials and Methods

### Synthesis of 2-acetamido-2-deoxy-1-thio-β-D-glucopyranose (1)

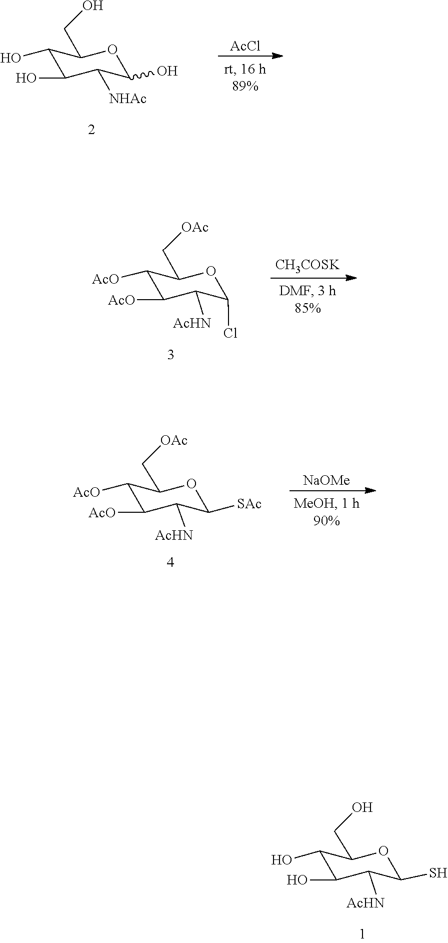

N-acetylglucosamine 2 was directly modified by treatment with acetyl chloride to yield the 1-chloro-substituted α-N-acetyl amino sugar 3. Nucleophilic substitution of the compound 3 with potassium thioacetate gave the corresponding 1-thiol-β-sugar 4. The thiol-sugar 4 was deacetylated with sodium methoxide affording the desired compound 1 with 90% yield.

### 2-Acetamido-2-deoxy-3,4,6-tri-O-acetyl-α-D-glucopyranosyl chloride (3)

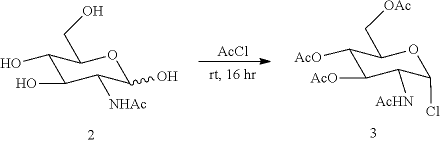 [SMILES: "AcCl" ; CC(=O)NC1C(O)OC(CO)C(O)C1O ; CC(=O)NC1C(Cl)OC(COC(C)=O)C(OC(C)=O)C1OC(C)=O]

2-Acetamido-2-deoxy-D-glucopyranose 2 (5.0 g, 22.62 mmol) was suspended in AcCl (8 mL, 12.44 mmol) under nitrogen atmosphere and the mixture was stirred at r.t. for 16 h. The reaction mixture was diluted with CH₂Cl₂ (50 mL) and extracted with ice water, saturated NaHCO₃, and brine solution. The organic layer was dried over Na₂SO₄, concentrated, and purified by silica gel flash column chromatography using ethyl acetate/hexane as eluent, affording compound 3 (7.40 g, 89%) as brown solid. ¹H NMR (400 MHz, CDCl₃) δ 6.20 (d, J=3.7 Hz, 1H), 5.88 (d, J=8.7 Hz, 1H), 5.39-5.29 (m, 1H), 5.23 (dd, J=12.0, 7.5 Hz, 1H), 4.55 (ddd, J=10.7, 8.8, 3.7 Hz, 1H), 4.32-4.24 (m, 2H), 4.15 (dd, J=13.1, 2.6 Hz, 1H), 2.12 (s, 3H), 2.07 (s, 6H), 2.00 (s, 3H); ¹³C NMR (100 MHz, CDCl₃) δ 171.50, 170.60, 170.13, 169.15, 93.65, 70.90, 70.14, 66.96, 61.15, 53.49, 23.10, 20.70, 20.56; HRMS (ESI): m/z Calcd for C₁₄H₂₀ClNO₈ [M]+: 365.0877; Found: 365.0758.

### 2,3,4,6-tetra-O-acetyl-1-S-acetyl-1-thio-β-D-glucopyranose (4)

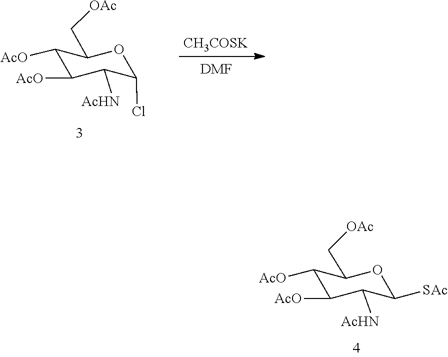

To the measured quantity of compound 3 (1 g, 2.73 mmol) in DMF (10 mL) at room temperature was added potassium thioacetate (1.56 g, 1.36 mmol) at nitrogen atmosphere and stirred for 3 h. Upon completion of reaction, ethyl acetate was added, and the resulting mixture was washed with water, saturated NaHCO₃, and brine solution. The organic layer was dried over Na₂SO₄, filtered, and concentrated. Purification was done by silica gel flash column chromatography using ethyl acetate/hexane (1:1) as eluent afforded compound 4 (0.94 g, 85%) as brown solid. ¹H NMR (400 MHz, CDCl₃) δ 5.76 (d, J=9.8 Hz, 1H), 5.14 (dt, J=10.4, 4.6 Hz, 2H), 4.37 (ddt, J=15.7, 10.6, 5.3 Hz, 1H), 4.26 (dd, J=12.5, 4.5 Hz, 1H), 4.12 (dd, J=12.5, 2.1 Hz, 1H), 3.86-3.78 (m, 1H), 2.39 (s, 3H), 2.10 (s, 3H), 2.06 (s, 6H), 1.94 (s, 3H); ¹³C NMR (101 MHz, CDCl₃) δ 193.68, 171.35, 170.74, 170.05, 169.24, 81.63, 76.57, 74.05, 67.74, 61.84, 52.19, 30.84, 23.15, 20.75, 20.67, 20.59; HRMS (ESI-TOF): Calcd. for C₁₆H₂₄NO₉S⁺: 406.1166; Found: 406.1114 (M+H⁺). See Alexander et al,. Org. Biomol. Chem., 15(10):2152-2156 (2017); Orth et al, Synthesis, 2010(13):2201-2206 (2010).

### 1-Thio-2-acetamido-2-deoxy-β-D-glucopyranose (1)

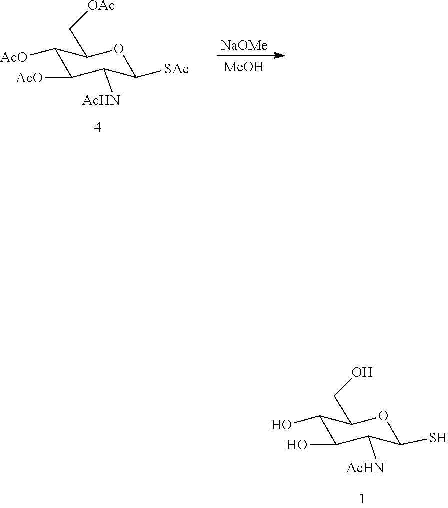

Compound 4 (0.5 g, 1.23 mmol) was dissolved in methanol (15 mL), added sodium methoxide (133 mg, 2.46 mmol), and stirred for 1 h, at which point TLC (ethyl acetate:methanol:water in 7:2:1 ratio) indicated complete consumption of starting material and formation of a single product. Dowex® 50WX8 (H+) ion exchange resin was added portion-wise until the reaction reached neutral pH. The mixture was then filtered and concentrated in vacuum. Purification was done by silica gel flash column chromatography using methanol/DCM (1:1) as eluent affording compound 1 (260 mg, 90%) as white solid. ¹H NMR (600 MHz, D₂O) δ 4.62 (d, J=10.2 Hz, 1H), 3.83 (d, J=12.3 Hz, 1H), 3.67 (dt, J=12.2, 7.1 Hz, 2H), 3.48-3.44 (m, 1H), 3.44-3.38 (m, 2H), 1.99 (s, 3H); ¹³C NMR (150 MHz, D₂O) δ 174.56, 80.20, 79.01, 74.95, 69.67, 60.81, 57.90, 22.31; HRMS (ESI-TOF): Calcd. for C₈H₁₅NO₅SNa⁺: 260.0563; Found: 260.0560 (M+Na⁺). See Alexander et al,. Org. Biomol. Chem., 15(10):2152-2156 (2017); Orth et al, Synthesis, 2010(13):2201-2206 (2010).

Plasmid Construction

pET-Duet-Afb_{4A}-7S-MBP-Z24TAG and pET-Duet-Afb_{4A}-7T-MBP-Z24TAG

To incorporate FSY into the Z protein and introduce Ser or Thr into the Afb protein for intermolecular GECCO, plasmids pET-Duet-Afb_{4A}-7S-MBP-Z24TAG plasmid and pET-Duet-Afb_{4A}-7T-MBP-Z24TAG plasmid were used for expression in E. coli, the generation of which was described previously. Wang et al, J. Am. Chem. Soc., 140:4995-4999 (2018).

pTak-sfGFP-182TAG-184S

To test intramolecular GECCO, FSY was incorporated into Tyr182 and Ser into Glu184 of sfGFP. Overlapping PCR was used to introduce the TAG codon and Ser codon into sfGFP gene, and the resultant PCR product was digested with Spe I and Blp I and ligated into the pTak vector pre-treated with the same restriction enzymes. Takimoto et al, ACS Chem. Biol. m 6:(7):733-743 (2011). pTak-sfGFP-NdeI-F is SEQ ID NO:26. pTak-sfGFP-Blp-R is SEQ ID NO:27. pTak-sfGFP-184S-F is SEQ ID NO:28. pTak-sfGFP-184S-R is SEQ ID NO:29.

pBad-Ub-45TAG

To test intramolecular GECCO, residue 45 of human ubiquitin was mutated into an amber stop codon TAG via overlapping PCR with following primers. PCR products were digested with Nde I and Hind III, and ligated into the commercial pBad vector pre-treated with the same restriction enzymes. pBAD-Ub-F is SEQ ID NO:30. pBAD-Ub-R is SEQ ID NO:31. Ub-45TAG-F is SEQ ID NO:32. Ub-45TAG-R: is SEQ ID NO:33.

pBad-Afb-37TAG

To test intramolecular GECCO, residue 37 of affibody was mutated into an amber stop codon TAG via site-directed mutagenesis with following primers. PCR products were digested with Nde I and Hind III, and ligated into the commercial pBad vector pre-treated with the same restriction enzymes. pBad-Afb-37TAG-F is SEQ ID NO:34. pBad-Afb-37TAG-R is SEQ ID NO:35.

Protein Expression

Afb_{4A}-7S, Afb_{4A}-7T, and MBP-Z24FSY

Plasmid pET-Duet-Afb_{4A}-7S-MBP-Z24TAG or pET-Duet-Afb_{4A}-7T-MBP-Z24TAG was co-transformed with plasmid pEvol-FSYRS into BL21(DE3) E. coli competent cells, respectively. Transformants were plated on an LB-Amp100Cm34 agar plate and incubated overnight at 37° C. A single colony was inoculated into 4 mL of 2×YT-Amp100Cm34 and agitated vigorously at 37° C. On the following day, overnight cell culture was diluted in 50 mL 2×YT-Amp100Cm34 at final OD₆₀₀ ˜0.1 and agitated vigorously at 37° C. When OD₆₀₀ reached 0.4, FSY compound was added to cell culture at final concentration of 1 mM. Cell culture was induced with 0.5 mM IPTG and 0.2% arabinose at OD₆₀₀˜0.5, and then incubated at 30° C. for 6 h. Cell pellets were collected by centrifugation at 2800 g for 10 min at 4° C. and stored at −80° C.

sfGFP-182FSY-184S

Plasmids pTak-sfGFP-182TAG-184S and pBK-FSYRS were co-transformed into DH10B E. coli competent cells. Transformants were plated on an LB-Kan50Cm34 agar plate and incubated overnight at 37° C. A single colony was inoculated into 4 mL of 2×YT-Kan50Cm34 and agitated vigorously at 37° C. On the following day, overnight cell culture was diluted in 50 mL 2×YT-Kan50Cm34 at final OD₆₀₀ —0.1 and agitated vigorously at 37° C. When OD₆₀₀ reached 0.4, cell culture was supplemented with 1 mM FSY. Cell culture was induced with 0.5 mM IPTG at OD₆₀₀˜0.5, then incubated at 30° C. for 6 h. Cell pellets were collected by centrifugation at 2800 g for 10 min at 4° C. and stored at −80° C.

Ub-45FSY and Afb-37FSY

Plasmids pBad-45TAG (or pBad-37TAG) and pEvol-FSYRS were co-transformed into DH10B E. coli competent cells. Transformants were plated on an LB-Amp100Cm34 agar plate and incubated overnight at 37° C. A single colony was inoculated into 4 mL of 2×YT-Amp100Cm34 and agitated vigorously at 37° C. On the following day, overnight cell culture was diluted in 50 mL 2×YT-Amp100Cm34 at final OD₆₀₀ ˜0.1 and agitated vigorously at 37° C. When OD₆₀₀ reached 0.4, cell culture was supplemented with 1 mM FSY. Cell culture was induced with 0.2% arabinose at OD₆₀₀˜0.5, then incubated at 30° C. for 6 h. Cell pellets were collected by centrifugation at 2800 g for 10 min at 4° C. and stored at −80° C.

His-Tag Protein Purification

Above cell pellets were resuspended in 14 mL lysis buffer (50 mM Tris-HCl pH 8.0, 500 mM NaCl, 20 mM imidazole, 1% v/v Tween 20, 10% v/v glycerol, lysozyme 1 mg/mL, DNase 0.1 mg/mL, and Roche protease inhibitor cocktails). The cell suspension was lysed at 4° C. for 30 min. Cell lysate was sonicated with Sonic Dismembrator (Fisher Scientific, 30% output, 3 min, 1 sec off, 1 sec on) in an ice-water bath, followed by centrifugation (20,000 g, 30 min, 4° C.). The soluble fractions were collected and incubated with pre-equilibrated Protino®Ni-NTA Agarose resin (400 μL) at 4° C. for 1 h with constant mechanical rotation. The slurry was loaded onto a Poly-Prep® Chromatography Column, washed with 5 mL of wash buffer (50 mM Tris-HCl pH 8.0, 500 mM NaCl, 20 mM imidazole, and 10% v/v glycerol) for 3 times, and eluted with 200 μL of elution buffer (50 mM Tris-HCl, pH 8.0, 500 mM NaCl, 250 mM imidazole, and 10% v/v glycerol) for 5 times. The eluates were concentrated and buffer exchanged into 100 μL of protein storage buffer (50 mM Tris-HCl, pH 7.4, and 150 mM NaCl) using Amicon Ultra columns, and stored at −80° C. for future analysis.

Ab Initio Folding of Afb

To study the conformational variability of the N-terminal region (Ser-1) of Afb, as well as that of Ser10, we performed ab initio folding of the Afb sequence prepended with Met(-3)Thr(-2)Ser(-1). We used the Rosetta program (Kaufmann et al, Biochemistry, 49:2987-2998 (2010)) to perform the folding simulations, getting 3- and 9-residue fragments (including homologs) from Robetta (Kim et al, Nucleic Acids Res., 32:W526-W531 (2004). The folding simulations were performed with the following command: ˜/rosetta_bin_linux_2018.33.60351_bundle/main/source/bin/AbinitioRelax.static.linuxgccrelease -database ˜/rosetta_bin_linux_2018.33.60351_bundle/main/database/ -in:file:frag3 aat000_03_05.200_v1_3 -in:file:frag9 aat000_09_05._200 v1_3 -abinitio:relax -relax:fast -abinitio::increase_cycles 10 -abinitio::rg_reweight 0.5 -abinitio::rsd_wt_helix 0.5 -abinitio::rsd_wt_loop 0.5 -use_filters true -psipred_ss2 frags_whom/t000_.psipred_ss2 -kill_hairpins t000_.psipred_ss2 -out:file:silent silent_$SGE_TASK_ID.out -nstruct 1 -evaluation:rmsd NATIVE_core afficore.txt -in:file:fasta affibody.fasta -in:file:native 1lp1ext.pdb -run:jran $SGE_TASK_ID -run:constant_seed -out:file:scorefile score_$SGE_TASK_ID.sc -out:path:all output_abinitio/ -out:user_tag $SGE_TASK_ID

Where the variable $SGE_TASK_ID varied from 1 to 10000 in increments of 1. The sequence folded was SEQ ID NO:36. The folded models were superimposed onto the affibody (1LP1, chain A) structure at residues 6-55 by Ca atoms.

To assess the effect of rotamer geometry on sfGFP residues 182 and 184, we approximated FSY as a sulfotyrosine. Residues of sulfotyrosine were collected from protein crystal structures deposited in the protein data bank, were aligned by N—C_{α}—C backbone atoms, and subsequently clustered by position of sidechain heavy atoms, where members within each cluster share an RMSD≤0.2 angstroms. The 252 cluster centroids (rotamers) of sulfotyrosine were superimposed onto residue 182 via the N—C_{α}—C backbone atoms, and those rotamers that clashed with the sfGFP protein were removed. Those rotamers that were accommodated by the sfGFP structure indicated that chemical interaction of FSY with Ser184 was geometrically hindered.

Labeling Dha-Containing sfGFP with 1-thiol-GlcNAc

Ten μg of sfGFP-182FSY-184Ser protein (expected to contain sfGFP-182Tyr-184Dha due to FSY conversion of Ser) expressed and purified from E. coli in 10 μL storage buffer were incubated with 300 mM 1-thiol-GlcNAc at 37° C. overnight. The same amount of purified sfGFP-128FSY-184E was used as the negative control. The reaction was terminated by acetone precipitation and then subject to MS and Western blot analysis.

Western Blot

After incubation with 1-thiol-GlcNAC, the sfGFP samples were separated on SDS-PAGE and immunoblotted with 1:1000 anti-GlcNAc monoclonal antibody followed by 1:10000 donkey anti-mouse secondary antibody to detect GlcNAc. An anti-His6 antibody was used to probe the C-terminally appended His-tag for loading control.

Protein Digestion and Peptide Desalting

Digestion of Afb_{4A}-7S, Afb_{4A}-7T, MBP-Z24FSY, Ub-45FSY, and Afb-37FSY

A total of 10 μg proteins of each sample in 10 μL storage buffer were digested by trypsin (at 50:1 protein:enzyme ratio) at 37° C. for 16 h. Digestion was stopped by adding formic acid to 5% final concentration, and digested peptides were desalted with StageTip.

Digestion of sfGFP-182FSY-184S

sfGFP-182FSY-184S proteins were heated at 98° C. for 10 min, and 10 μg of proteins in storage buffer were digested with trypsin (at 50:1 protein:enzyme ratio) at 37° C. for 16 h. Digestion was stopped by adding formic acid to 5% final concentration, and digested peptides were desalted with StageTip.

Digestion of GlcNAc Labelled sfGFP-182FSY-184S Protein

A total of 10 μg proteins were precipitated by six volumes of acetone at −20° C. for 30 min. Precipitated proteins were dried in air and resuspended in storage buffer. Sample solution was heated at 98° C. for 10 min, and 10 μg of proteins in storage buffer were digested with trypsin (at 50:1 protein:enzyme ratio) at 37° C. for 16 h. Digestion was stopped by adding formic acid to 5% final concentration, and digested peptides were desalted with StageTip.

Tandem Mass Spectrometric Analysis

For Afb_{4A}-7S, Afb_{4A}-7T, and MBP-Z24FSY samples, digested peptides were analyzed with an in-line EASY-spray source and nano-LC UltiMate 3000 high-performance liquid chromatography system (Thermo Fisher) interfaced with Elite mass spectrometer (Thermo Fisher). Peptides were eluted over gradient of 2%-40% buffer B (80% acetonitrile, 20% H₂O, 0.1% formic acid) at flow rate 300 nL/min from EASY-Spray PepMap C18 Columns (50 cm; particle size, 2 μm; pore size, 100 Å; Thermo Fisher). For different samples, slight modifications were made to the separation method. The Elite mass spectrometer was operated in data-dependent mode with one full MS scan at R=60,000 (m/z=200) mass range from 375 to 1800 (AGC target 1×10⁶), followed by ten CID MS/MS scans. A dynamic exclusion time of 30 s was used, and singly charged ions were excluded.

All other samples were performed using an Orbitrap Fusion Lumos™ instrument (ThermoFisher, San Jose, Calif.) coupled with an UltiMate™ 3000 nano LC. Mobile phase A and B were water and acetonitrile, respectively, with 0.1% formic acid. Protein digests were loaded directly onto a C18 PepMap EASYspray column (ThermoFisher Scientific, part number ES803) at a flow rate of 300 nL/min. Peptides were separated using a linear gradient of 2% to 40% B over 38 min. Survey scans of peptide precursors were performed from 375 to 1500 m/z at 60,000 FWHM resolution with a 4×10⁵ ion count target and a maximum injection time of 50 ms. The instrument was set to run in top speed mode with 3 second cycles for the survey and the MS/MS scans. After a survey scan, tandem MS was then performed on the most abundant precursors exhibiting a charge state from 2 to 7 of greater than 5×10⁴ intensity by isolating them in the quadrupole at 1.6 Da. Higher energy collisional dissociation (HCD) fragmentation was applied with 30% collision energy and resulting fragments detected in the Orbitrap detector at a resolution of 30,000. The maximum injection time limited was 50 ms and dynamic exclusion was set to 60 seconds with a 10 ppm mass tolerance around the precursor.

Informal equence Listing

SEQ ID NO: 1 (amino acid sequence of FSYR)
MDKKPLNTLISATGLWMSRTGTIHKIKHHEVSRSKIYIEMACGDHLVVNNSRSSRTARAL
RHHKYRKTCKRCRVSDEDLNKFLTKANEDQTSVKVKVVSAPTRTKKAMPKSVARAPKPLE
NTEAAQAQPSGSKFSPAIPVSTQESVSVPASVSTSISSISTGATASALVKGNTNPITSMS
APVQASAPALTKSQTDRLEVLLNPKDEISLNSGKPFRELESELLSRRKKDLQQIYAEERE
NYLGKLEREITRFFVDRGFLEIKSPILIPLEYIERMGIDNDTELSKQIFRVDKNFCLRPM
LIPNLYNYLRKLDRALPDPIKIFEIGPCYRKESDGKEHLEEFTMLTFIQMGSGCTRENLE
SIITDFLNHLGIDFKIVGDSCMVLGDTLDVMHGDLELSSAVVGPIPLDREWGIDKPKIGA
GFGLERLLKVKHDFKNIKRAARSESYYNGISTNL*
SEQ ID NO: 2 (nucleic acid (DNA) sequence of FSYR)
ATGGATAAAAAGCCTTTGAACACTCTGATTTCTGCGACCGGTCTGTGGATGTCCCGCACCGGCA
CCATCCACAAAATCAAACACCATGAAGTTAGCCGTTCCAAAATCTACATTGAAATGGCTTGCGG
CGATCACCTGGTTGTCAACAACTCCCGTTCTTCTCGTACCGCTCGCGCACTGCGCCACCACAAA
TATCGCAAAACCTGCAAACGTTGCCGTGTTAGCGATGAGGACCTGAACAAATTCCTGACCAAAG
CTAACGAGGATCAGACCTCCGTAAAAGTGAAGGTAGTAAGCGCTCCGACCCGTACTAAAAAGGC
TATGCCAAAAAGCGTGGCCCGTGCCCCGAAACCTCTGGAAAACACCGAGGCGGCTCAGGCTCAA
CCATCCGGTTCTAAATTTTCTCCGGCGATCCCAGTGTCCACCCAAGAATCTGTTTCCGTACCAG
CAAGCGTGTCTACCAGCATTAGCAGCATTTCTACCGGTGCTACCGCTTCTGCGCTGGTAAAAGG
TAACACTAACCCGATTACTAGCATGTCTGCACCGGTACAGGCAAGCGCCCCAGCTCTGACTAAA
TCCCAGACGGACCGTCTGGAGGTGCTGCTGAACCCAAAGGATGAAATCTCTCTGAACAGCGGCA
AGCCTTTCCGTGAGCTGGAAAGCGAGCTGCTGTCTCGTCGTAAAAAGGATCTGCAACAGATCTA
CGCTGAGGAACGCGAGAACTATCTGGGTAAGCTGGAGCGCGAAATTACTCGCTTCTTCGTGGAT
CGCGGTTTCCTGGAGATCAAATCTCCGATTCTGATTCCGCTGGAATACATTGAACGTATGGGCA
TCGATAATGATACCGAACTGTCTAAACAGATCTTCCGTGTGGATAAAAACTTCTGTCTGCGTCC
GATGCTGATTCCGAACTTGTACAACTATTTACGTAAACTGGACCGTGCCCTGCCGGACCCGATC
AAAATATTCGAGATCGGTCCTTGCTACCGTAAAGAGTCCGACGGTAAAGAGCACCTGGAAGAAT
TCACCATGCTGACATTCATTCAGATGGGTAGCGGTTGCACGCGTGAAAACCTGGAATCCATTAT
CACCGACTTCCTGAATCACCTGGGTATCGATTTCAAAATTGTTGGTGACAGCTGTATGGTGTTA
GGCGATACGCTGGATGTTATGCACGGCGATCTGGAGCTGTCTTCCGCAGTTGTGGGCCCAATCC
CGCTGGATCGTGAGTGGGGTATCGACAAACCTAAAATCGGTGCGGGTTTTGGTCTGGAGCGTCT
GCTGAAAGTAAAACACGACTTCAAGAACATCAAACGTGCTGCACGTTCCGAGTCCTATTACAAT
GGTATTTCTACTAACCTGTAA
SEQ ID NO: 3 (wild-type amino acid sequence of Methanosarcina mazei PylRS)
MDKKPLNTLISATGLWMSRTGTIHKIKHHEVSRSKIYIEMACGDHLVVNNSRSSRT
ARALRHHKYRKTCKRCRVSDEDLNKFLTKANEDQTSVKVKVVSAPTRTKKAMPKSV
ARAPKPLENTEAAQAQPSGSKFSPAIPVSTQESVSVPASVSTSISSISTGATASAL
VKGNTNPITSMSAPVQASAPALTKSQTDRLEVLLNPKDEISLNSGKPFRELESELL
SRRKKDLQQIYAEERENYLGKLEREITRFFVDRGFLEIKSPILIPLEYIERMGIDN
DTELSKQIFRVDKNFCLRPMLAPNLYNYLRKLDRALPDPIKIFEIGPCYRKESDGK
EHLEEFTMLNFCQMGSGCTRENLESIITDFLNHLGIDFKIVGDSCMVYGDTLDVMH
GDLELSSAVVGPIPLDREWGIDKPWIGAGFGLERLLKVKHDFKNIKRAARSESYYNGISTNL*
SEQ ID NO: 4 (nucleic acid sequence of tRNA_{CUA}^{Pyl}))
ggaaacctgatcatgtagatcgaatggactctaaatccgttcagccgggttagattcccggggtttccg
SEQ ID NO: 5
CTAACAGGAGGAATTAGATCTATGGATAAAAAGCCT
SEQ ID NO: 6
GATGATGATGATGATGGTCGACTTACAGGTTAGTAGAA
SEQ ID NO: 7
TATGCCATGGATAAAAAGCCTTTG
SEQ ID NO: 8
CTATGCTAGCTTACAGGTTAGTAGA
SEQ ID NO: 9
AACGCGGAACTATCAGTCGCCGGC
SEQ ID NO: 10
AACAAAGAACTATCAGTCGCCGGC
SEQ ID NO: 11
AACTGCGAACTATCAGTCGCCGGC
SEQ ID NO: 12
AACAGCGAACTATCAGTCGCCGGC
SEQ ID NO: 13
AACACCGAACTATCAGTCGCCGGC
SEQ ID NO: 14
AACCATGAACTATCAGTCGCCGGC
SEQ ID NO: 15
GAACGCGTTGTCTACCATGGTATATCTCC
SEQ ID NO: 16 is
CCATGGTAGACAACGCGTTCAACTATGAACTATCAGTCGCC
SEQ ID NO: 17 is
TATATCTCCTTCTTAAAGTTAAACAAAATTATTTCTAGAGGGG
SEQ ID NO: 18
AACTATGACTAGTCATGACCAACTGAC
SEQ ID NO: 19
CGCATACGCGTCCGCCTACGCTCTAGCCATCATAGT
SEQ ID NO: 20
TGGCTAGAGCGTAGGCGGACGCGTATGCGGAAGAGGAAATCCG
SEQ ID NO: 21
CCAAGCTCAGCTTATTAGTGATGGTGATG
SEQ ID NO: 22
TATACATATGTCCAAACTCGATCTAAACG
SEQ ID NO: 23
AGCCAAGCTTTTAATGATGATGATGATGATGCCCTTCGTGTAACCCACATTCC
SEQ ID NO: 24
GAACATCGATTAGAACCCTGGCAC
SEQ ID NO: 25
AGTTTTGCAACGGTCAGTTTG
SEQ ID NO: 26 is pTak-sfGFP-NdeI-F:
GAGGAGAAATTACATATGAGCAAGGGCGAGGAG
SEQ ID NO: 27 is pTak-sfGFP-Blp-R:
CCAAGCTCAGCTTAGTGATGGTGATGGTGATGGAGCTCCTTGTACAGCTC
SEQ ID NO: 28 is pTak-sfGFP-184S-F:
CGCCGACCACTAGCAGTCTAACACCCCCATCGGC
SEQ ID NO: 29 is pTak-sfGFP-184S-R:
GCCGATGGGGGTGTTAGACTGCTAGTGGTCGGCG
SEQ ID NO: 30 pBAD-Ub-F:
ATCGCATATGCAGATCTTTGTGAAGACCCTCA
SEQ ID NO: 31 is pBAD-Ub-R:
CGATAAGCTTTTAATGATGATGATGATGATGCCCACCTCGCAGGC
SEQ ID NO: 32 is Ub-45TAG-F:
TGACCAGCAGCGTCTGATATAGGCCGGCAAACAGCTGG
SEQ ID NO: 33 is Ub-45TAG-R:
CCAGCTGTTTGCCGGCCTATATCAGACGCTGCTGGTCA
SEQ ID NO: 34 is pBad-Afb-37TAG-F:
TTTATGGGATTAGCCAAGCCAAAG
SEQ ID NO: 35: pBad-Afb-37TAG-R:
CTGAAGATGAAGGCCTTC
SEQ ID NO: 36:
MTSVDNKFNKELSVAGREIVTLPNLNDPQKKAFIFSLWDDPSQSANLLAEAK
KLNDAQAPK

What is claimed is:

1. A method of converting an amino acid to a chemically reactive amino acid, the method comprising: (i) contacting an FSY protein with the amino acid; thereby converting the amino acid to a chemically reactive amino acid.

2. The method of claim 1, further comprising glycosylating the reactive amino acid.

3. The method of claim 1, wherein the amino acid is serine and the chemically reactive amino acid is dehydroalanine.

4. The method of claim 1, wherein the amino acid is threonine and the chemically reactive amino acid is dehydrobutyrine.

5. The method of claim 1, wherein contacting comprises a sulfur-fluoride exchange reaction.

6. The method of claim 5, wherein contacting comprises a proximity-enabled, sulfur-fluoride exchange reaction.

7. The method of claim 1, wherein the FSY protein comprises the amino acid.

8. The method of claim 7, wherein the amino acid is proximal to the fluorosulfate-L-tyrosine in the FSY protein.

9. The method of claim 1, wherein the method comprises contacting the FSY protein with a second protein comprising the amino acid.

10. The method of claim 7, wherein the amino acid and the fluorosulfate-L-tyrosine in the FSY protein are in a protein α-helix.

11. The method of claim 7, wherein the amino acid and the fluorosulfate-L-tyrosine in the FSY protein are in a protein β-strand.

12. The method of claim 7, wherein the amino acid and the fluorosulfate-L-tyrosine in the FSY protein are in a protein loop.

13. The method of claim 1, wherein the contacting is performed within a cell.

14. The method of claim 13, wherein the cell is a bacterial cell.

15. The method of claim 13, wherein the cell is a mammalian cell.

16. The method of claim 1, further comprising, prior to the contacting in step (i), performing the step: (ii) contacting a protein, a pyrrolysyl-tRNA synthetase, a tRNA^{Pyl}, and a fluorosulfate-L-tyrosine, thereby forming the FSY protein.

17. A protein comprising:
(a) (i) fluorosulfate-L-tyrosine, and (ii) serine, threonine, or a combination thereof proximal to the fluorosulfate-L-tyrosine;
(b) (i) fluorosulfate-L-tyrosine, and (ii) dehydroalanine, dehydrobutyrine, or a combination thereof proximal to the fluorosulfate-L-tyrosine; or
(c) (i) tyrosine, and (ii) dehydroalanine, dehydrobutyrine, or a combination thereof proximal to the tyrosine.

18. A protein complex comprising:
(a) (i) a first protein comprising fluorosulfate-L-tyrosine, and (ii) a second protein comprising serine, threonine, or a combination thereof; wherein the fluorosulfate-L-tyrosine in the first protein is proximal to the serine, threonine, or the combination thereof in the second protein;
(b) (i) a first protein comprising fluorosulfate-L-tyrosine, and (ii) a second protein comprising dehydroalanine, dehydrobutyrine, or a combination thereof; wherein the fluorosulfate-L-tyrosine in the first protein is proximal to the dehydroalanine, dehydrobutyrine, or the combination thereof in the second protein; or
(c) (i) a first protein comprising tyrosine, and (ii) a second protein comprising dehydroalanine, dehydrobutyrine, or a combination thereof; wherein the tyrosine in the first protein is proximal to the dehydroalanine, dehydrobutyrine, or the combination thereof in the second protein.


SEQUENCE LISTING (36 sequences)

SEQ ID NO: 1 - protein, 454 aa, Artificial Sequence
  Synthetic polypeptide
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLIPNLYNYLRKLDRALPDPIKIFEIGPCYRKESDGKEHLEEFTMLTFIQMGSGCTRENLESIITDFLNHLGIDFKIVGDSCMVLGDTLDVMHGDLELSSAVVGPIPLDREWGIDKPKIGAGFGLERLLKVKHDFKNIKRAARSESYYNGISTNL

SEQ ID NO: 2 - DNA, 1365 bp, Artificial Sequence
  Synthetic polynucleotide
atggataaaaagcctttgaacactctgatttctgcgaccggtctgtggatgtcccgcaccggcaccatccacaaaatcaaacaccatgaagttagccgttccaaaatctacattgaaatggcttgcggcgatcacctggttgtcaacaactcccgttcttctcgtaccgctcgcgcactgcgccaccacaaatatcgcaaaacctgcaaacgttgccgtgttagcgatgaggacctgaacaaattcctgaccaaagctaacgaggatcagacctccgtaaaagtgaaggtagtaagcgctccgacccgtactaaaaaggctatgccaaaaagcgtggcccgtgccccgaaacctctggaaaacaccgaggcggctcaggctcaaccatccggttctaaattttctccggcgatcccagtgtccacccaagaatctgtttccgtaccagcaagcgtgtctaccagcattagcagcatttctaccggtgctaccgcttctgcgctggtaaaaggtaacactaacccgattactagcatgtctgcaccggtacaggcaagcgccccagctctgactaaatcccagacggaccgtctggaggtgctgctgaacccaaaggatgaaatctctctgaacagcggcaagcctttccgtgagctggaaagcgagctgctgtctcgtcgtaaaaaggatctgcaacagatctacgctgaggaacgcgagaactatctgggtaagctggagcgcgaaattactcgcttcttcgtggatcgcggtttcctggagatcaaatctccgattctgattccgctggaatacattgaacgtatgggcatcgataatgataccgaactgtctaaacagatcttccgtgtggataaaaacttctgtctgcgtccgatgctgattccgaacttgtacaactatttacgtaaactggaccgtgccctgccggacccgatcaaaatattcgagatcggtccttgctaccgtaaagagtccgacggtaaagagcacctggaagaattcaccatgctgacattcattcagatgggtagcggttgcacgcgtgaaaacctggaatccattatcaccgacttcctgaatcacctgggtatcgatttcaaaattgttggtgacagctgtatggtgttaggcgatacgctggatgttatgcacggcgatctggagctgtcttccgcagttgtgggcccaatcccgctggatcgtgagtggggtatcgacaaacctaaaatcggtgcgggttttggtctggagcgtctgctgaaagtaaaacacgacttcaagaacatcaaacgtgctgcacgttccgagtcctattacaatggtatttctactaacctgtaa

SEQ ID NO: 3 - protein, 454 aa, Methanosarcina mazeii
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 4 - DNA, 69 bp, Methanosarcina mazeii
ggaaacctgatcatgtagatcgaatggactctaaatccgttcagccgggttagattcccggggtttccg

SEQ ID NO: 5 - DNA, 36 bp, Artificial Sequence
  Synthetic polynucleotide
ctaacaggaggaattagatctatggataaaaagcct

SEQ ID NO: 6 - DNA, 38 bp, Artificial Sequence
  Synthetic polynucleotide
gatgatgatgatgatggtcgacttacaggttagtagaa

SEQ ID NO: 7 - DNA, 24 bp, Artificial Sequence
  Synthetic polynucleotide
tatgccatggataaaaagcctttg

SEQ ID NO: 8 - DNA, 25 bp, Artificial Sequence
  Synthetic polynucleotide
ctatgctagcttacaggttagtaga

SEQ ID NO: 9 - DNA, 24 bp, Artificial Sequence
  Synthetic polynucleotide
aacgcggaactatcagtcgccggc

SEQ ID NO: 10 - DNA, 24 bp, Artificial Sequence
  Synthetic polynucleotide
aacaaagaactatcagtcgccggc

SEQ ID NO: 11 - DNA, 24 bp, Artificial Sequence
  Synthetic polynucleotide
aactgcgaactatcagtcgccggc

SEQ ID NO: 12 - DNA, 24 bp, Artificial Sequence
  Synthetic polynucleotide
aacagcgaactatcagtcgccggc

SEQ ID NO: 13 - DNA, 24 bp, Artificial Sequence
  Synthetic polynucleotide
aacaccgaactatcagtcgccggc

SEQ ID NO: 14 - DNA, 24 bp, Artificial Sequence
  Synthetic polynucleotide
aaccatgaactatcagtcgccggc

SEQ ID NO: 15 - DNA, 29 bp, Artificial Sequence
  Synthetic polynucleotide
gaacgcgttgtctaccatggtatatctcc

SEQ ID NO: 16 - DNA, 41 bp, Artificial Sequence
  Synthetic polynucleotide
ccatggtagacaacgcgttcaactatgaactatcagtcgcc

SEQ ID NO: 17 - DNA, 43 bp, Artificial Sequence
  Synthetic polynucleotide
tatatctccttcttaaagttaaacaaaattatttctagagggg

SEQ ID NO: 18 - DNA, 27 bp, Artificial Sequence
  Synthetic polynucleotide
aactatgactagtcatgaccaactgac

SEQ ID NO: 19 - DNA, 36 bp, Artificial Sequence
  Synthetic polynucleotide
cgcatacgcgtccgcctacgctctagccatcatagt

SEQ ID NO: 20 - DNA, 43 bp, Artificial Sequence
  Synthetic polynucleotide
tggctagagcgtaggcggacgcgtatgcggaagaggaaatccg

SEQ ID NO: 21 - DNA, 29 bp, Artificial Sequence
  Synthetic polynucleotide
ccaagctcagcttattagtgatggtgatg

SEQ ID NO: 22 - DNA, 29 bp, Artificial Sequence
  Synthetic polynucleotide
tatacatatgtccaaactcgatctaaacg

SEQ ID NO: 23 - DNA, 53 bp, Artificial Sequence
  Synthetic polynucleotide
agccaagcttttaatgatgatgatgatgatgcccttcgtgtaacccacattcc

SEQ ID NO: 24 - DNA, 24 bp, Artificial Sequence
  Synthetic polynucleotide
gaacatcgattagaaccctggcac

SEQ ID NO: 25 - DNA, 21 bp, Artificial Sequence
  Synthetic polynucleotide
agttttgcaacggtcagtttg

SEQ ID NO: 26 - DNA, 33 bp, Artificial Sequence
  Synthetic polynucleotide
gaggagaaattacatatgagcaagggcgaggag

SEQ ID NO: 27 - DNA, 50 bp, Artificial Sequence
  Synthetic polynucleotide
ccaagctcagcttagtgatggtgatggtgatggagctccttgtacagctc

SEQ ID NO: 28 - DNA, 34 bp, Artificial Sequence
  Synthetic polynucleotide
cgccgaccactagcagtctaacacccccatcggc

SEQ ID NO: 29 - DNA, 34 bp, Artificial Sequence
  Synthetic polynucleotide
gccgatgggggtgttagactgctagtggtcggcg

SEQ ID NO: 30 - DNA, 32 bp, Artificial Sequence
  Synthetic polynucleotide
atcgcatatgcagatctttgtgaagaccctca

SEQ ID NO: 31 - DNA, 45 bp, Artificial Sequence
  Synthetic polynucleotide
cgataagcttttaatgatgatgatgatgatgcccacctcgcaggc

SEQ ID NO: 32 - DNA, 38 bp, Artificial Sequence
  Synthetic polynucleotide
tgaccagcagcgtctgatataggccggcaaacagctgg

SEQ ID NO: 33 - DNA, 38 bp, Artificial Sequence
  Synthetic polynucleotide
ccagctgtttgccggcctatatcagacgctgctggtca

SEQ ID NO: 34 - DNA, 24 bp, Artificial Sequence
  Synthetic polynucleotide
tttatgggattagccaagccaaag

SEQ ID NO: 35 - DNA, 18 bp, Artificial Sequence
  Synthetic polynucleotide
ctgaagatgaaggccttc

SEQ ID NO: 36 - protein, 61 aa, Artificial Sequence
  Synthetic polypeptide
MTSVDNKFNKELSVAGREIVTLPNLNDPQKKAFIFSLWDDPSQSANLLAEAKKLNDAQAPK